(19) United States
(12) Patent Application Publication
(10) Pub. No.: US 2016/0026009 A1
(43) Pub. Date: Jan. 28, 2016

(54) GLASSES FOR AMBLYOPIA TREATMENT

(71) Applicants: Nejc URBAJS, Hrastnik (SI); Ben-Ezra OMRY, Tel-Aviv (IL); Andrej TOMELJAK, Ljubljana (SI); Tjasa VRHOVNIK, Borovnica (SI); Uros JAHV, Ljubljana (SI); Jure REPE, Zgornje Gorje (SI); Matic MEKINDA, Ljubljana (SI); Marjan PLEGER, Miklavz pri Ormozu (SI); Domen GASVODA, Ljubljana (SI); Luka STEPAN, Kranj (SI); Vilijem FUCHS, Dol pri Ljubljani (SI); Ami DROR, Tel Aviv (IL); Todd MATTINGLY, Huston,, TX (US); X6D LIMITED, Limassol (CY)

(72) Inventors: Nejc URBAJS, Hrastnik (SI); Ben-Ezra OMRY, Tel-Aviv (IL); Andrej TOMELJAK, Ljublijana (SI); Tjasa VRHOVNIK, Borovnica (SI); Uros JAVH, Ljubljana (SI); Jure REPE, Zgornje Gorje (SI); Matic MEKINDA, Lubljana (SI); Marjan PLEGER, Miklavz pri Omozu (SI); Domen GAZVODA, Ljubljana (SI); Luka STEPAN, Kranj (SI); Vilijem FUCHS, Dol pri Ljubljana (SI); Ami DROR, Tel-Aviv (IL); Todd MATTINGLY, Huston, TX (US)

(21) Appl. No.: 14/648,901

(22) PCT Filed: Oct. 30, 2013
(86) PCT No.: PCT/US13/67371

§ 371 Date: Jun. 1, 2015

Related U.S. Application Data
(60) Provisional application No. 61/721,105, filed on Nov. 1, 2012.

(51) Int. Cl. G02C 7/16; G02F 1/13; A61F 9/04; G02C 9/00; G02C 11/00; G02B 27/22; G02F 1/133

(52) U.S. Cl. CPC G02C 7/16; G02B 27/2264; G02F 1/1313; G02F 1/13306; G02C 9/00; G02C 11/10; A61F 9/04

(57) ABSTRACT

A system for treating Lazy Eye that includes shutter glasses. During treatment, one of the shutters of the glasses are controllably cycled between and open and closed state.

### 1. CROSS-REFERENCE TO RELATED APPLICATIONS

This application claims the benefit of the filing date of U.S. Provisional Patent Application No. 61/721,105, attorney docket number 092847.001555, filed on Nov. 1, 2012, the disclosure of which is incorporated herein by reference.

This application claims the benefit of the filing date of U.S. Provisional Patent Application No. 61/641,594, attorney docket no. 092847.001519, filed on May 2, 2012, the disclosure of which is incorporated herein by reference.

This application claims the benefit of the filing date of U.S. Provisional Patent Application No. 61/678,301, attorney docket no. 092847.001535, filed on Aug. 1, 2012, the disclosure of which is incorporated herein by reference.

This application claims the benefit of the filing date of U.S. Provisional Patent Application No. 61/347,243, attorney docket no. 092847.000333, filed on May 21, 2010, the disclosure of which is incorporated herein by reference.

This application claims the benefit of the filing date of U.S. Provisional Patent Application No. 61/447,801, attorney docket no. 092847.000884, filed on Mar. 1, 2011, the disclosure of which is incorporated herein by reference.

This application is a continuation-in-part of U.S. utility patent application Ser. No. 13/113,784, attorney docket no. 092847.001066, filed on May 23, 2011, which claimed the benefit of the filing date of U.S. Provisional Patent Application No. 61/347,243, attorney docket no. 092847.000333, filed on May 21, 2010, the disclosures of which is incorporated herein by reference.

This application is a continuation-in-part of PCT patent application no. PCT/US11/5100, attorney docket no. 092847.001289, filed on Sep. 9, 2011, which claimed the benefit of the filing date of U.S. Provisional Patent Application No. 61/381,329, attorney docket no. 092847.00518, filed on Sep. 9, 2010, the disclosures of which is incorporated herein by reference.

This application is a continuation-in-part of PCT patent application no. PCT/US12/20123, attorney docket no. 092847.001318, filed on Jan. 4, 2012, which claimed the benefit of the filing date of U.S. Provisional Patent Application No. 61/429,689, attorney docket no. 092847.000778, filed on Jan. 4, 2011, the disclosures of which is incorporated herein by reference.

This application is a continuation-in-part of U.S. utility patent application Ser. No. 13/019,896, attorney docket no. 092847.000254, filed on Feb. 2, 2011, which claimed the benefit of the filing dates of each of: U.S. provisional patent application serial No. 61/337,392, attorney docket no. 092847.000242, filed on Feb. 3, 2010, U.S. Provisional Patent Application No. 61/337,470, attorney docket no. 092847.000243, filed on Feb. 4, 2010, U.S. Provisional Patent Application No. 61/337,565, attorney docket no. 092847.000244, filed on Feb. 8, 2010, and U.S. Provisional Patent Application No. 61/307,287, attorney docket no. 092847.000253, filed on Feb. 23, 2010, the disclosures of which are incorporated herein by reference.

This application is a continuation-in-part of U.S. Utility patent application Ser. Nos. 12/619,518, attorney docket no. 092847.000027; 12/619,517, attorney docket no. 092847.000042; 12/619,309, attorney docket no. 092847.000043; 12/619,415, attorney docket no. 092847.000044; 12/619,400, attorney docket no. 092847.000045; 12/619,431, attorney docket no. 092847.000046; 12/619,163, attorney docket no. 092847.000060; 12/619,456, attorney docket no. 092847.000064; 12/619,102, attorney docket no. 092847.000080, all filed on Nov. 16, 2009, and all of which claim the benefit of the filing date of U.S. Provisional Patent Application No. 61/179,248, attorney docket no. 092847.000020, filed on May 18, 2009, and the filing date of U.S. Provisional Patent Application No. 61/115,477, attorney docket no. 092847.000008, filed on Nov. 17, 2008, the disclosures of all which are incorporated herein by reference.

This application claims the benefit of the filing date of U.S. Provisional Patent Application No. 61/680,908, attorney docket no. 092847.001538, filed on Aug. 8, 2012, the disclosure of which is incorporated herein by reference.

### 2. BACKGROUND

This disclosure relates to image processing systems for the controllable presentation of an image to a viewer.

### BRIEF DESCRIPTION OF THE DRAWINGS

FIG. 1 is a block diagram of an exemplary embodiment of 3D shutter glasses.

FIG. 2 is an illustration of the 3D shutter glasses of FIG. 1.

FIG. 3 is an exploded view of the 3D shutter glasses of FIG. 2.

FIG. 4 is an illustration of a portion of the 3D shutter glasses of FIG. 2.

FIGS. 5a, 5b and 5c are illustrations of various aspects of the 3D shutter glasses of FIG. 2.

FIGS. 6a, 6b and 6c are illustrations of various aspects of the 3D shutter glasses of FIG. 2.

FIGS. 7a, 7b and 7c are illustrations of various aspects of the 3D shutter glasses of FIG. 2.

FIGS. 8a, 8b, 8c and 8d are illustrations of various aspects of the 3D shutter glasses of FIG. 2.

FIGS. 9a and 9b are flow chart illustrations of an exemplary embodiment of a method for operating the 3D shutter glasses of FIGS. 1-8d.

FIGS. 10a and 10b are flow chart illustrations of an exemplary embodiment of a method for operating the 3D shutter glasses of FIGS. 1-8d.

FIGS. 11a-11c are flow chart illustrations of an exemplary embodiment of a method for operating the 3D shutter glasses of FIGS. 1-8d.

FIGS. 12a-12b are flow chart illustrations of an exemplary embodiment of a method for operating the 3D shutter glasses of FIGS. 1-8d.

FIG. 13 is a flow chart illustration of an exemplary embodiment of a method for operating the 3D shutter glasses of FIGS. 1-8d.

FIGS. 14a-14h are illustrations of an exemplary embodiment of shutter glasses for the treatment of Lazy Eye.

FIGS. 15a and 15b are flow chart illustrations of an exemplary embodiment of operating shutter glasses to provide sunglasses.

FIG. 16 is a flow chart illustration of an exemplary embodiment of operating shutter glasses to treat Intermittent Exotropia.

FIG. 17 is a flow chart illustration of an exemplary embodiment of a method of adjusting the operating parameters of a pair of shutter glasses.

FIG. 18 is a flow chart illustration of an exemplary embodiment of a method of providing parental control of a pair of shutter glasses.

FIG. 19 is a schematic illustration of an exemplary embodiment of a system for treating Amblyopia.

### DETAILED DESCRIPTION

In the drawings and description that follows, like parts are marked throughout the specification and drawings with the same reference numerals, respectively. The drawings are not necessarily to scale. Certain features of the invention may be shown exaggerated in scale or in somewhat schematic form and some details of conventional elements may not be shown in the interest of clarity and conciseness. The present invention is susceptible to embodiments of different forms. Specific embodiments are described in detail and are shown in the drawings, with the understanding that the present disclosure is to be considered an exemplification of the principles of the invention, and is not intended to limit the invention to that illustrated and described herein. It is to be fully recognized that the different teachings of the embodiments discussed below may be employed separately or in any suitable combination to produce desired results. The various characteristics mentioned above, as well as other features and characteristics described in more detail below, will be readily apparent to those skilled in the art upon reading the following detailed description of the embodiments, and by referring to the accompanying drawings.

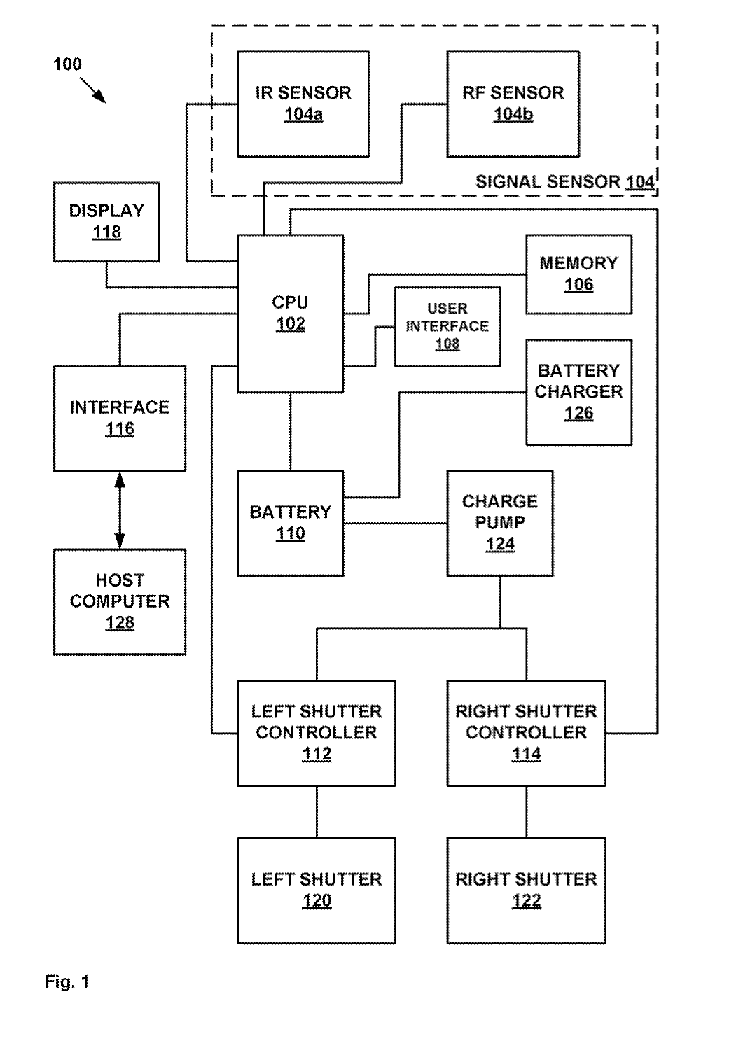

Referring now to FIG. 1, an exemplary embodiment of 3D shutter glasses 100 includes a CPU 102 that is operably coupled to a signal sensor 104, a memory 106, a user interface 108, a battery 110, a left shutter controller 112, a right shutter controller 114, an interface 116, and a display 118. In an exemplary embodiment, the signal sensor 104 may include an infrared (“IR”) sensor 104a and/or a radiofrequency (“RF”) sensor 104b. In an exemplary embodiment, the RF sensor 104b may include a sensor capable of communications with Bluetooth, or other RF communication standards, enabled devices.

In an exemplary embodiment, left and right shutters, 120 and 122, respectively, are operably coupled to the left and right shutter controllers, 112 and 114, respectively, for controlling the operation of the left and right shutters. In an exemplary embodiment, as will be recognized by persons having ordinary skill in the art, the left and right shutters, 120 and 122, are controlled by the left and right shutter controllers, 112 and 114, to adjust the degree of optical transmissivity of the respective shutters during operation of the 3D shutter glasses. In an exemplary embodiment, the left and right shutters, 120 and 122, may include pi cells and/or twisted nematic (“TN”) cells and/or other conventional liquid crystal cells, or equivalents thereof. As a result, a wearer of the 3D glasses 100 is able to view 3D content such as, for example, 3D movies, 3D TV, and 3D gaming images. In an exemplary embodiment, during operation, the left and right shutters, 120 and 122, may be controlled by the left and right shutter controllers, 112 and 114, to adjust the degree of optical transmissivity of the respective shutters during operation of the 3D shutter glasses as a function of one or more external signals transmitted to and sensed by the signal sensor 104.

In an exemplary embodiment, a charge pump 124 may also be operably coupled to the battery and the left and right shutter controllers, 112 and 114, for providing increased current and or charge and or voltage to the left and right shutters, 120 and 122, during the operation of the 3D glasses 100.

In an exemplary embodiment, a conventional battery charger 126 may also be operably coupled to the battery 110 for recharging the battery.

In an exemplary embodiment, the interface 116 is capable of interfacing with a host computer 128 which may, for example, include a laptop or desktop computer, smart-phone, or equivalent devices, the Internet, or other network, or equivalent devices.

The general design and operation of the 3D glasses 100, for viewing 3D images, except as described herein below, are considered well known to persons having ordinary skill in the art, and the general design and operation of the 3D glasses 100, for viewing 3D images is described and illustrated in one or more of the following: U.S. Pat. No. 8,233,103, the disclosure of which is incorporated herein by reference.

In an exemplary embodiment, the memory 106 includes a conventional non volatile read/write memory device and the user interface 108 includes one or more conventional user input and user interface devices such as, for example, push button, touch screen, displays or indicator lights.

Furthermore, the interface 116 includes one or more conventional interface devices such as, for example, a USB, a WiFi, a Bluetooth or other wired and/or wireless interface and the display 118 includes one or more conventional display devices such as, for example, an LED, an LCD, a plasma or other display device. In an exemplary embodiment, the interface 116 may include one or more conventional wired or wireless connections or interfaces which are capable of connecting the 3D glasses to one or more host computers 128 or wired and/or wireless networks.

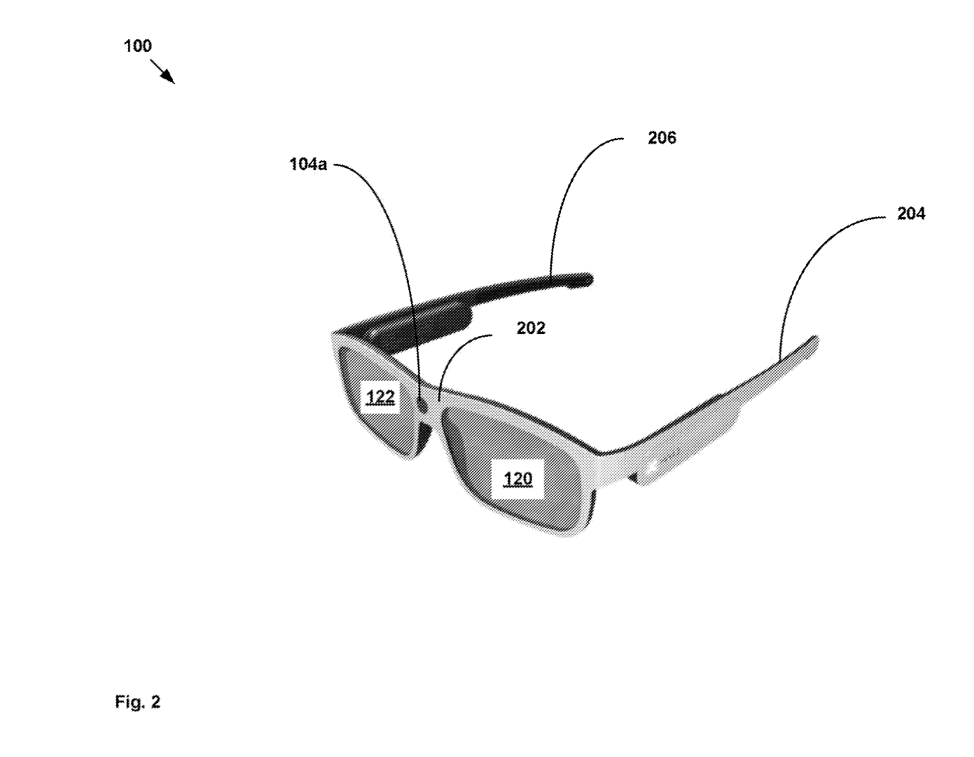

Referring now to FIG. 2, in an exemplary embodiment, the 3D shutter glasses 100 include a front frame 202 for mounting the left and right shutters, 120 and 122, and the IR sensor 104a, and left and right temples, 204 and 206, coupled to and extending from the front frame.

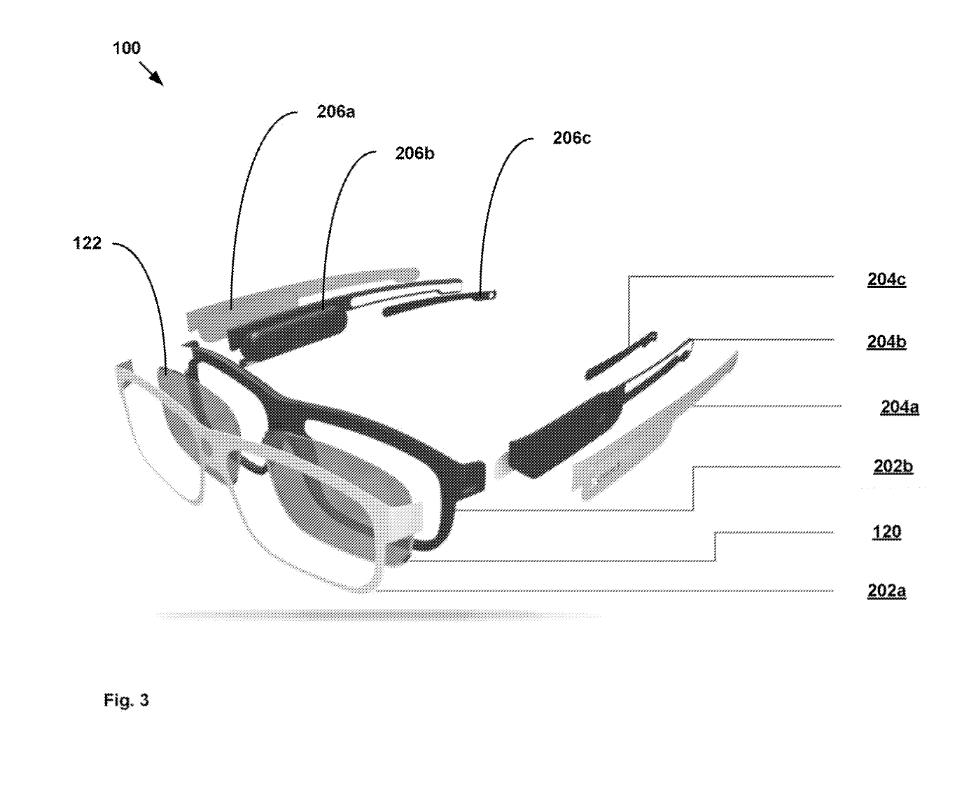

Referring now to FIG. 3, in an exemplary embodiment, the front frame 202 includes an outer front frame 202a and an inner front frame 202b for supporting the left and right shutters, 120 and 122. In an exemplary embodiment, the left temple 204 includes an outer left temple 204a, an inner left temple 204b, and an inner left temple rubber member 204c. In an exemplary embodiment, the right temple 206 includes an outer right temple 206a, an inner right temple 206b, and an inner right temple rubber member 206c.

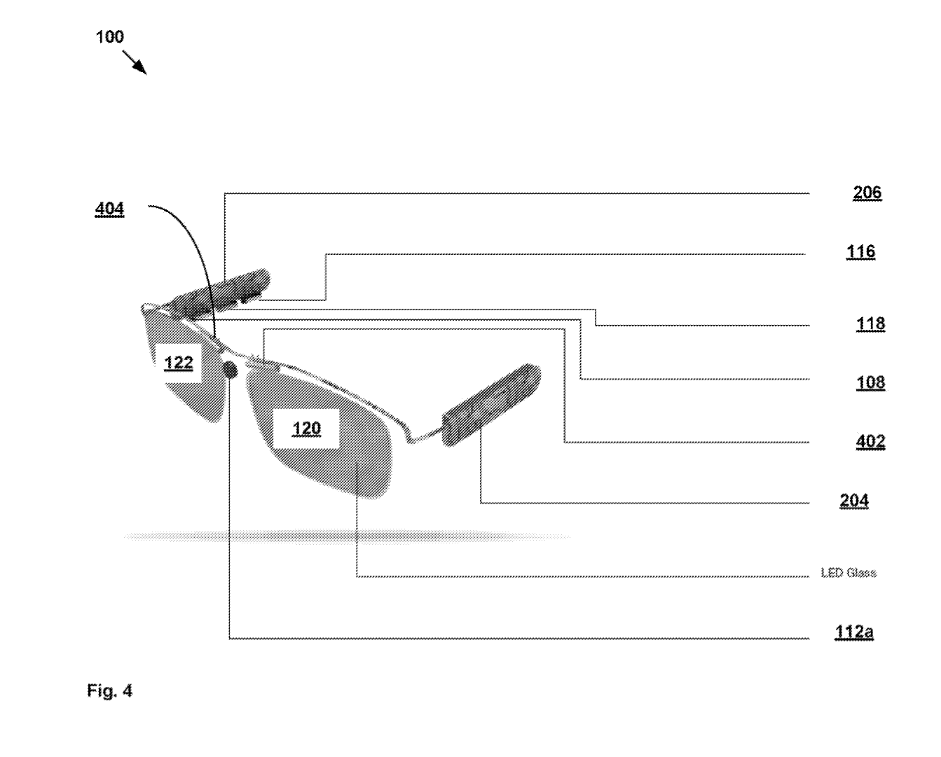

Referring now to FIG. 4, in an exemplary embodiment, the left and right shutters, 120 and 122, are operably coupled to the CPU 102 by left and right connections, 402 and 404. As illustrated, in an exemplary embodiment, the circuitry of the 3D shutter glasses 100 are positioned within and coupled to the left and right temples, 204 and 206. In this manner, the volume and mass of the front frame 202 of the 3D shutter glasses 100 is reduced. Furthermore, in an exemplary embodiment, the user interface 108 includes one or more visual indicators and is coupled to the control circuitry positioned within and coupled to the right temple 206. Furthermore, in an exemplary embodiment, the display 118 includes one or more display devices and is coupled to the control circuitry positioned within and coupled to the right temple 206. Furthermore, in an exemplary embodiment, the interface 116 permits the battery 110 of the 3D shutter glasses 100 to be re-charged and the CPU 102 of the 3D shutter glasses to be operably coupled to one or more host computers, smart phones, personal digital assistants and/or the Internet and is coupled to the control circuitry positioned within and coupled to the right temple 206.

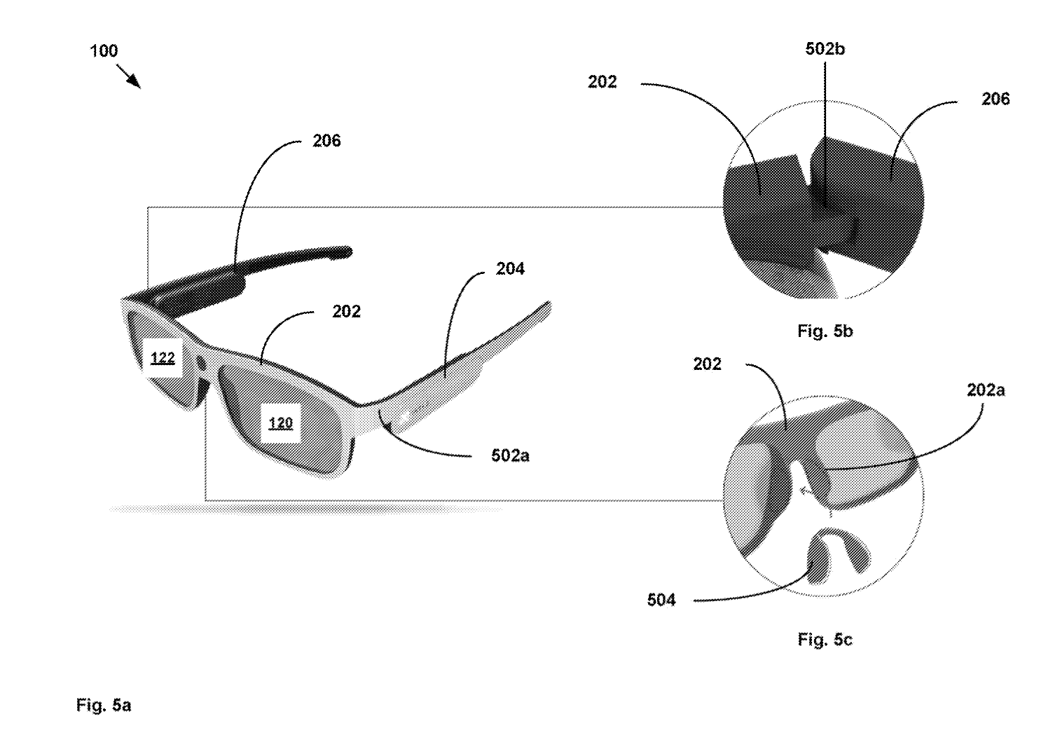

Referring now to FIGS. 5a, 5b and 5c, in an exemplary embodiment, the left and right temples, 204 and 206, are pivotally coupled to the front frame 202 by left and right hinges, 502a and 502b. In an exemplary embodiment, the 3D shutter glasses 100 further include removable nose pieces 504 that are adapted to resiliently and releasably engage the nose piece 202a of the front frame 202.

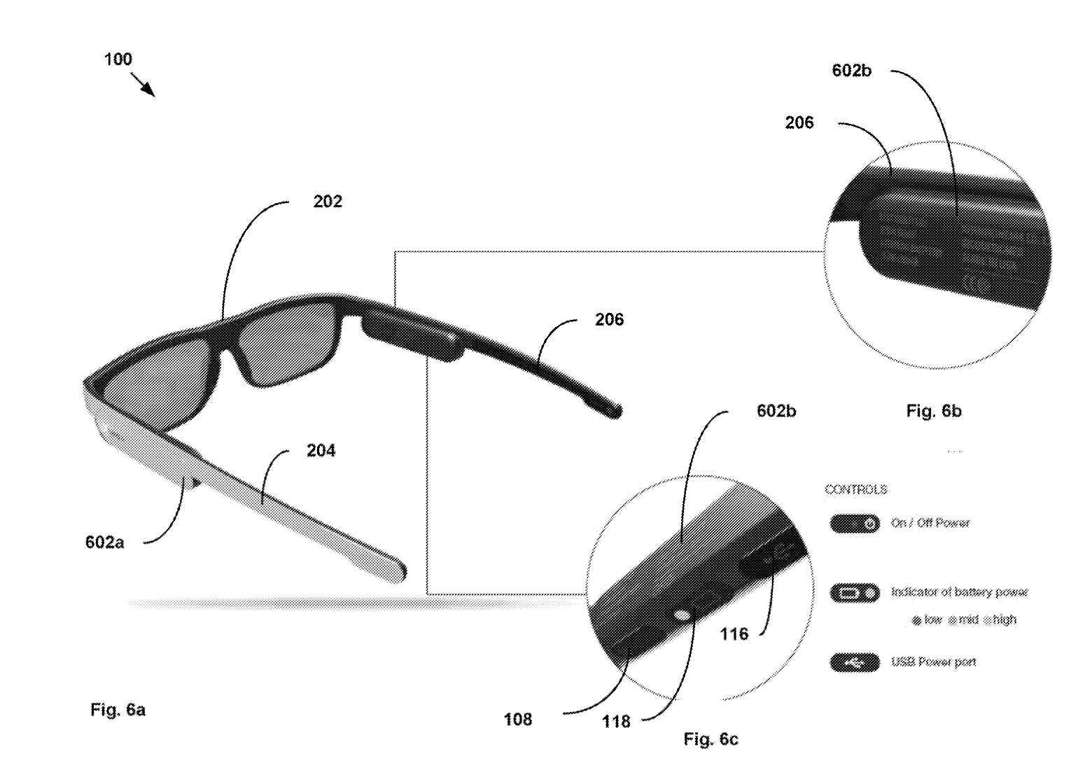

Referring now to FIGS. 6a, 6b and 6c, in an exemplary embodiment, each of the left and right temples, 204 and 206, include elongated housings, 602a and 602b, for housing at least a portion of the control system for the 3D shutter glasses 100. Furthermore, in an exemplary embodiment, the user interface 108, the display 118 and the interface 116 are operably coupled to the housing 602b.

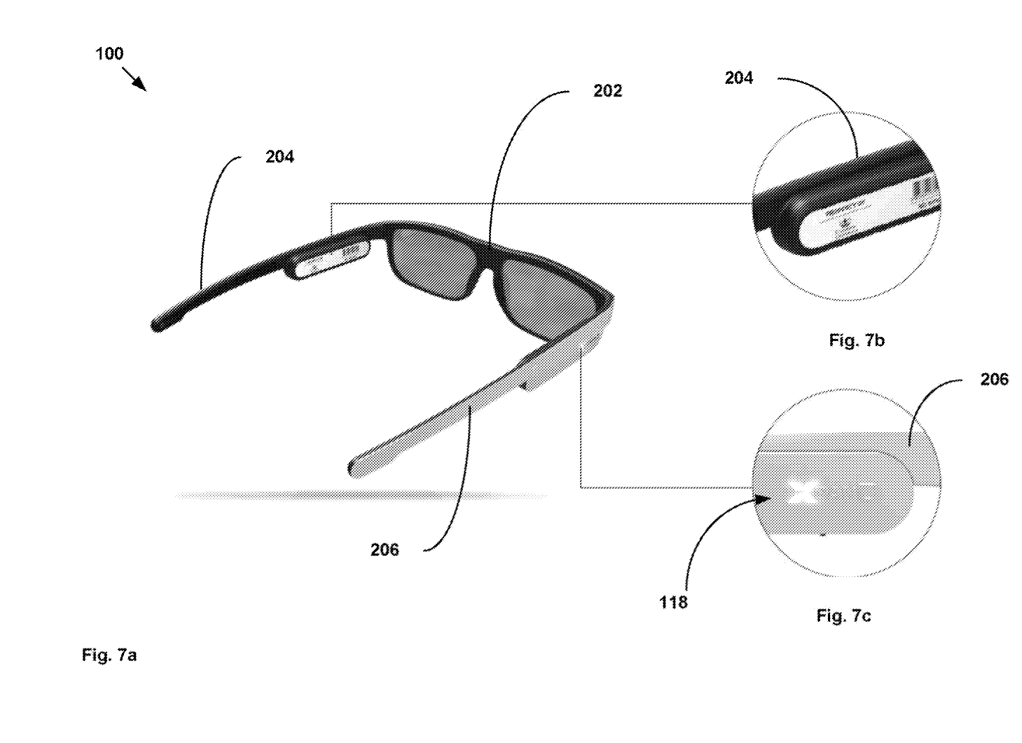

Referring now to FIGS. 7a, 7b, and 7c, in an exemplary embodiment, one or both of the left and right temples, 204 and 506, include a display 118 that may, for example, include a light that glows for a predetermined period when, for example, the 3D shutter glasses 100 are initially turned on, the operating mode of the 3D shutter glasses changes, or some other circumstance.

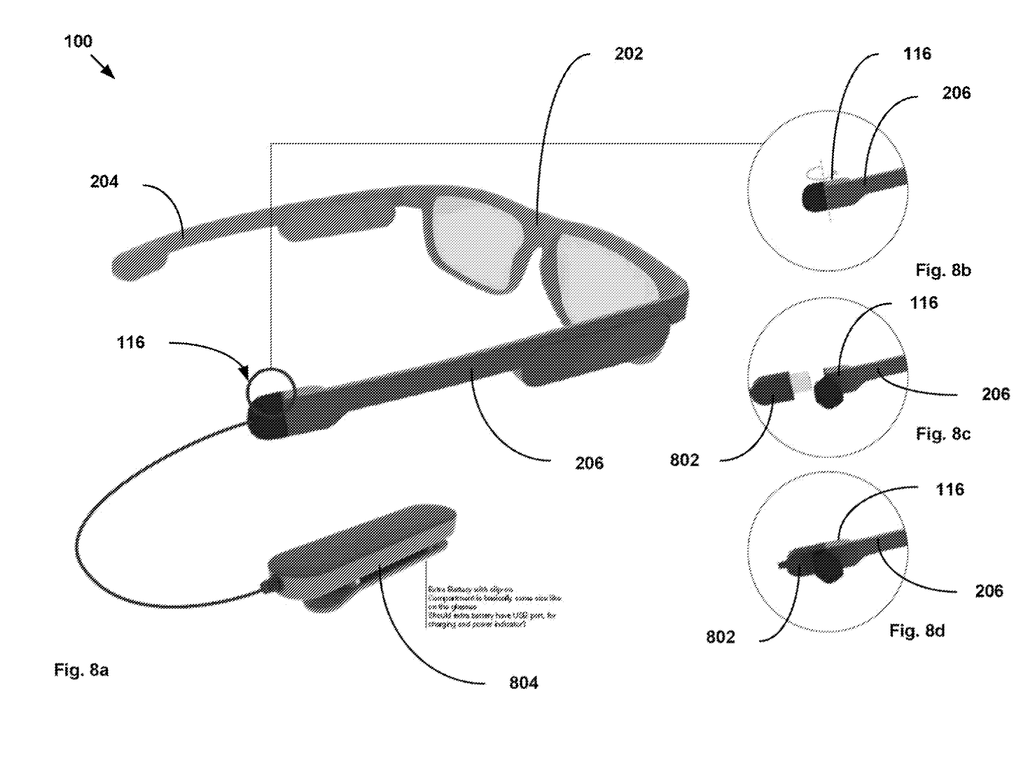

Referring now to FIGS. 8a, 8b, 8c and 8d, in an exemplary embodiment, one or both of the left and right temples, 204 and 206, include the interface 116 that permits the 3D shutters glasses 100 to be operably coupled to one or more host computers 128 using a conventional connection such as, for example, a USB connection 802. In an exemplary embodiment, the interface 116 may also be used to connect an external battery 804 to the 3D shutter glasses 100 for providing electrical power thereto. In an exemplary embodiment, the interface 116 may also be used to connect a battery charger to the 3D shutter glasses 100 for charging the battery 110.

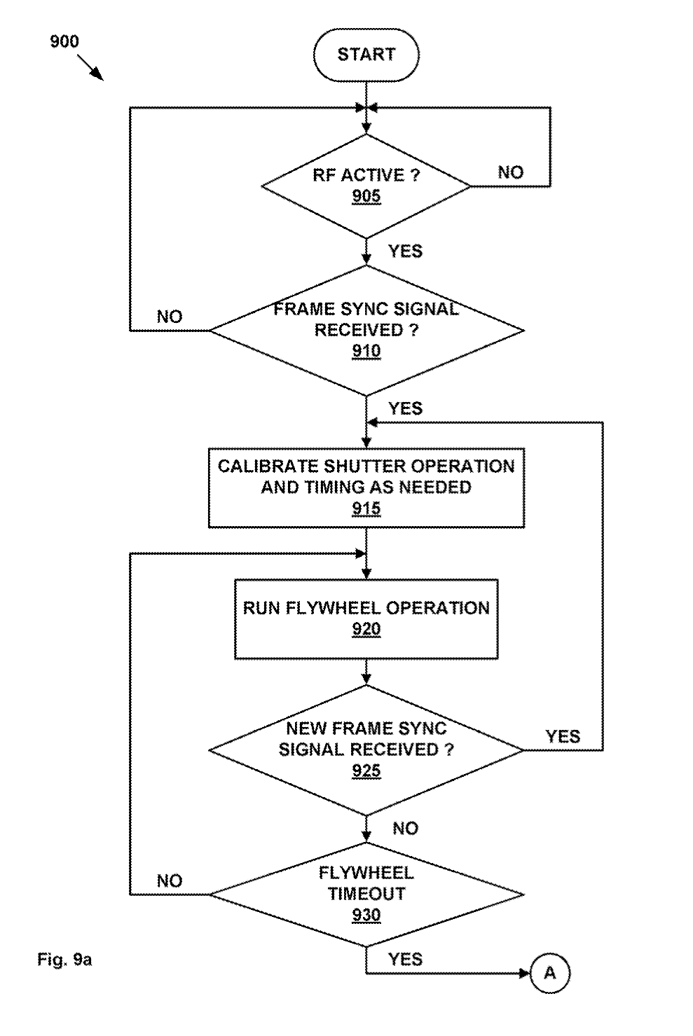

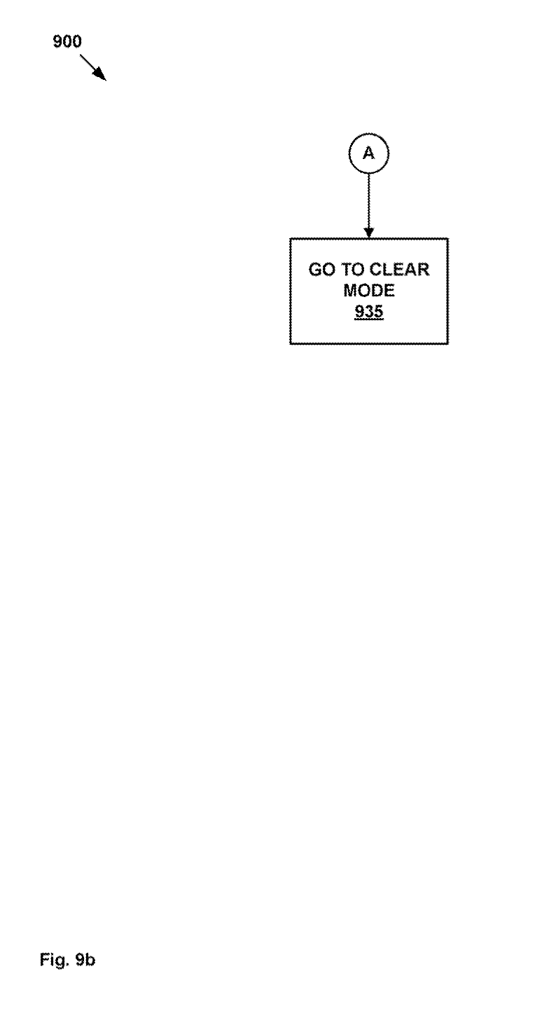

Referring now to FIGS. 9a and 9b, in an exemplary embodiment, the 3D shutter glasses 100 implement a method of operation in which, in 905, the 3D shutter glasses determine if a radio frequency (“RF”) signal has been received. If the 3D shutter glasses determine that a RF signal has been received, then, in 910, the 3D shutter glasses determine if the RF signal includes a frame synchronization signal. In an exemplary embodiment, the frame synchronization signal may include one or indicia representative of one or more operating parameters for viewing a frame displayed on a display device operably coupled to the 3D shutter glasses 100.

If the RF signal includes a frame synchronization signal, then, in 915, the 3D glasses 100 may calibrate and/or adjust the timing of the operation of the left and right shutters, 120 and 122, accordingly. The 3D shutter glasses 100 may then run a flywheel operation of the left and right shutters, 120 and 122, in 920. In an exemplary embodiment, during the flywheel operation, the left and right shutters, 120 and 122, are alternatively opened and closed at an operating frequency that is a function of the content of the frame synchronization signal.

If the 3D shutter glasses 100 receive a new frame synchronization signal in 925, then the 3D shutter glasses will then repeat 915, 920 and 925. Alternatively, if the 3D shutter glasses 100 do not receive a new frame synchronization signal in 925, then the 3D shutter glasses will determine if a timeout of the flywheel mode of operation has occurred in 930. If the 3D shutter glasses 100 do not determine that a timeout of the flywheel mode of operation occurred in 930, then the 3D shutter glasses will then repeat 920, 925 and 930. Alternatively, if the 3D shutter glasses 100 determine that a timeout of the flywheel mode of operation occurred in 930, then the 3D shutter glasses will then go to a clear mode of operation in 935. In an exemplary embodiment, in 935, the 3D shutter glasses 100 are operated in a clear mode of operation such that the left and right shutters, 120 and 120, are both optically transparent such that the wearer of the glasses sees a 2D image on a display device. In an exemplary embodiment, in 935, the 3D shutter glasses 100 are operated in a clear mode of operation.

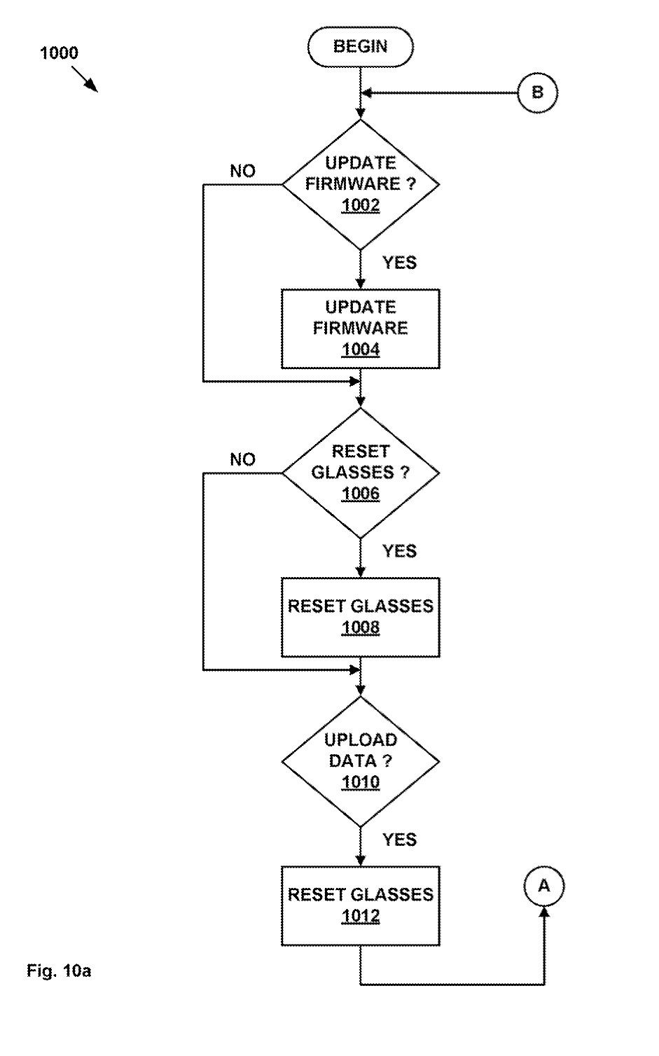

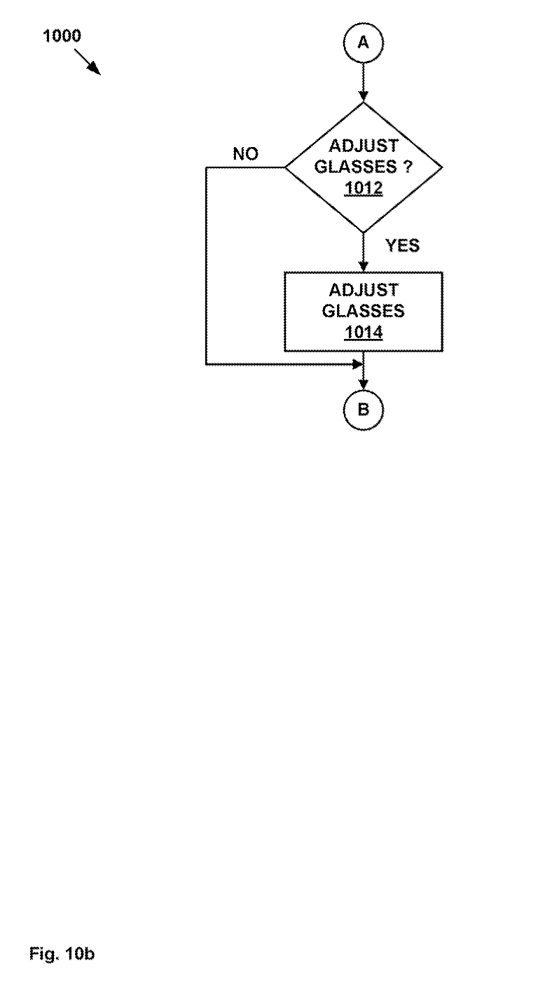

Referring now to FIGS. 10a and 10b, in an exemplary embodiment, the 3D shutter glasses 100 implement a method of operation 1000 in which the 3D shutter glasses are operably coupled to one or more of the host computers 128.

In 1002 and 1004, the user may operate one or more of the host computers 128 to update the firmware of the 3D shutter glasses 100. In this manner, the operation of the 3D shutter glasses 100 may be changed by each user of the 3D shutter glasses.

In 1006 and 1008, the user may operate one or more of the host computers 128 to reset the firmware of the 3D shutter glasses 100. In this manner, the operation of the 3D shutter glasses 100 may be reset by each user of the 3D shutter glasses.

In 1010 and 1012, the user may operate one or more of the host computers 128 to upload data from the 3D shutter glasses 100 to one or more of the host computers, or other computers operably coupled to one or more of the host computers. In this manner, the data regarding the status and/or operation of the 3D shutter glasses 100 may be uploaded by each user of the 3D shutter glasses.

In 1012 and 1014, the user may operate one or more of the host computers 128 to adjust one or more operating parameters of the 3D shutter glasses 100. In this manner, each user may customize the operation of the 3D shutter glasses 100.

In an exemplary embodiment, the 3D glasses 100 may be operated to provide a treatment for persons having Amblyopia (also commonly referred to as “Lazy Eye”). The general method of operating shutter glasses, such as the 3D glasses 100, for the treatment of Lazy Eye is considered well known to persons having ordinary skill in the art and is disclosed, for example, in U.S. Pat. No. 5,452,026, the disclosure of which is incorporated herein by reference.

Thus, the 3D glasses 100 may be operated to treat Lazy Eye by operating the shutters, 120 and 122, to provide an intermittent eye occluder. As a result, the 3D glasses may be used to replace traditional treatments for treating Lazy Eye such as, for example, occlusion of the sound eye, by patching over the sound eye, or penalization modality using atropine drops for blurring the vision of the sound eye. In this manner, the 3D glasses 100 may be operated to treat Lazy Eye by providing repetitive occlusion-transparent sessions on the sound eye by shuttering the sound eye using the corresponding shutter at a selected frequency while permitting the weak eye to view through the other substantially optically transparent shutter.

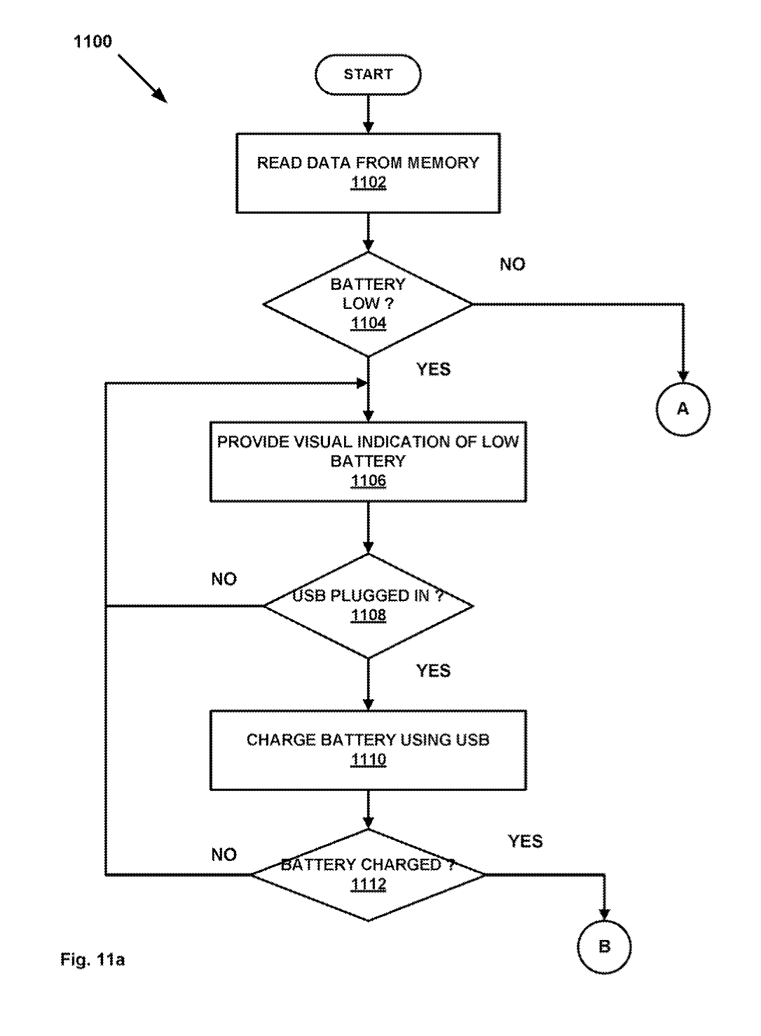

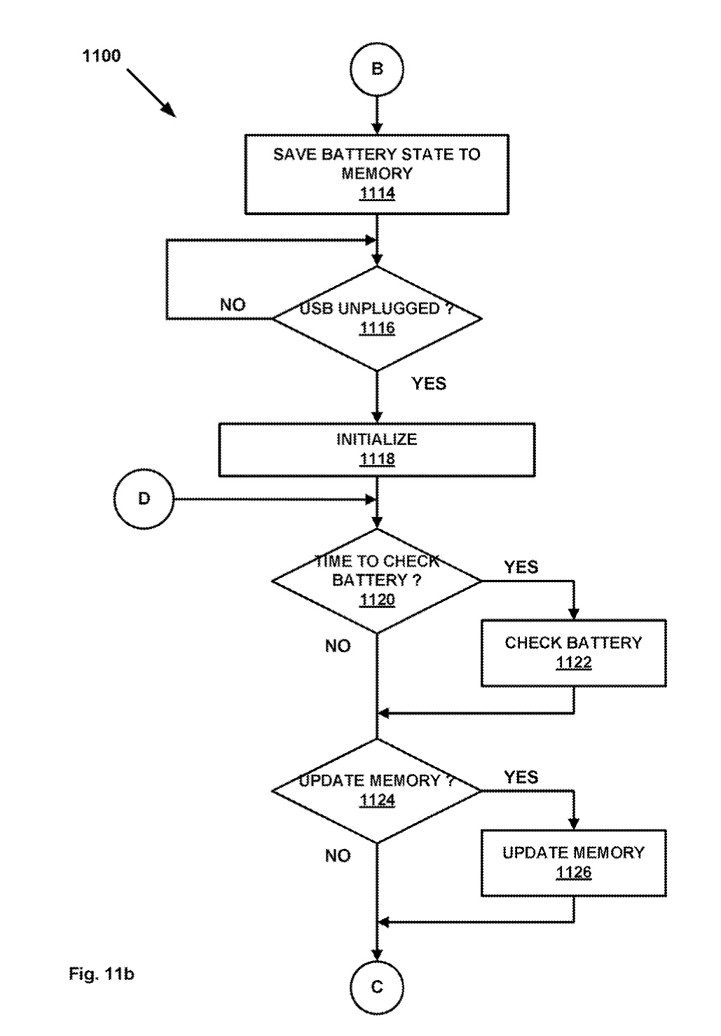

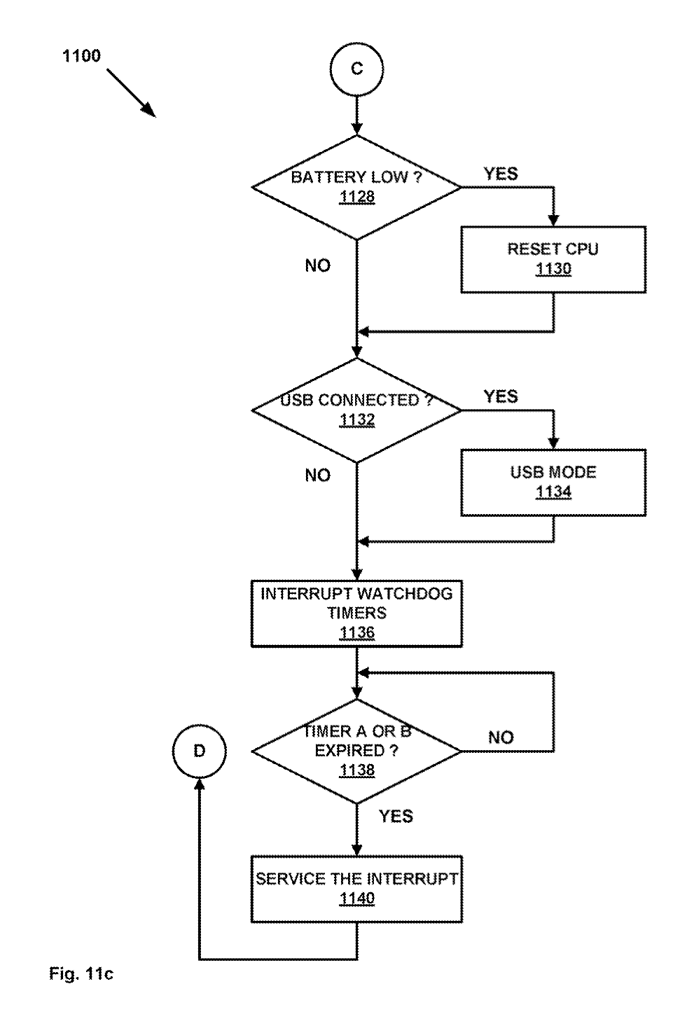

Referring to FIGS. 11a-11c, in an exemplary embodiment, the 3D glasses 100 may be operated in accordance with a method 1100 in which, in 1102, data is read from the 106. In an exemplary embodiment, the data that is read from the memory 106 may include, for example, operational instructions for operating the 3D glasses 100 which may include the present method of operation and/or other operating parameters that may define certain aspects of the operational characteristics of the 3D glasses.

In 1104, the 3D glasses 100 then determine if the remaining power in the battery 110 is low. If the battery level is low, then, in 1106, a visual indication of the low battery level is provided in 1106 by, for example, operating the user interface 108 and/or display 118 to provide such visual indication of a low battery to a user of the 3D glasses 100.

In 1108, the 3D glasses 100 then determine if a USB connection has been made to the interface 116. If a USB connection has been made, then, in 1110, the battery 110 may be charged using the USB connection. If the battery 110 has been charged in 1112, then the operating state of the battery 110 is saved to the memory 106 in 1114. The 3D glasses 100 then determine if the USB connection has been removed in 1116. If the USB connection has been removed, then the 3D glasses 100 initialize the CPU 102.

In 1120, the 3D glasses 100 then determine if it is time to check the operating state of the battery 110. If it is time to check the operating state of the battery 110, then, in 1122, the 3D glasses 100 check the operating state of the battery and, the power level of the battery is low, then steps 1106 to 1118 may be repeated in order to charge the battery.

In 1124, the 3D glasses 100 then determine if the memory 106 should be updated. If the 3D glasses 100 determine that the memory 106 should be updated, then in 1126, the 3D glasses 100 permit the memory to be updated in 1126. In an exemplary embodiment, the contents of the memory 106 may be updated by using the interface 116 to connect the 3D glasses to a host computer 128.

In 1128, the 3D glasses 100 determine if the power level of the battery 110 is low. If the power level of the battery 106 is low, then, in 1130, the 3D glasses 100 may reset the CPU 102.

In 1132, the 3D glasses 100 determine if a USB connection has been made to the interface 116. If a USB connection has been made, then the 3D glasses 100 may operate in a USB mode of operation in 1134. In an exemplary embodiment, the USB mode of operation may also operate whenever the 3D glasses 100 are connected to one or more host computers 128 and/or networks, through a wired and/or wireless connection, via the interface 116.

In 1136, the 3D glasses 100 may initiate one or more interrupt watchdog timers. In 1138, if one or more of the interrupt watchdog timers have expired, then the 3D glasses may service one or more of the interrupts in 1140.

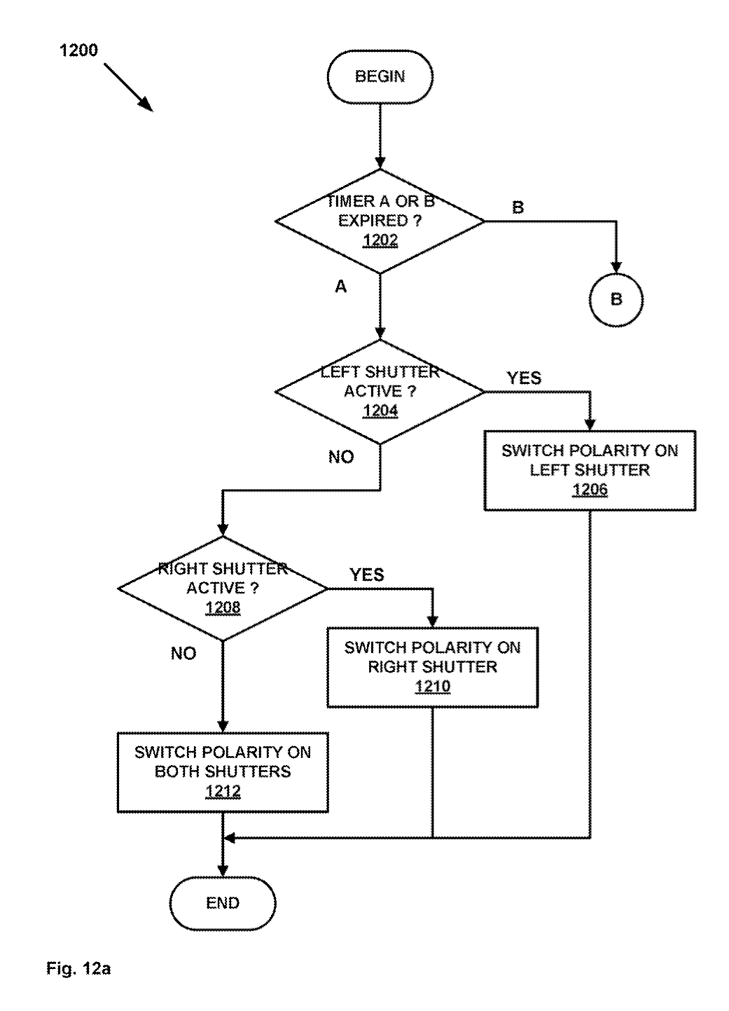

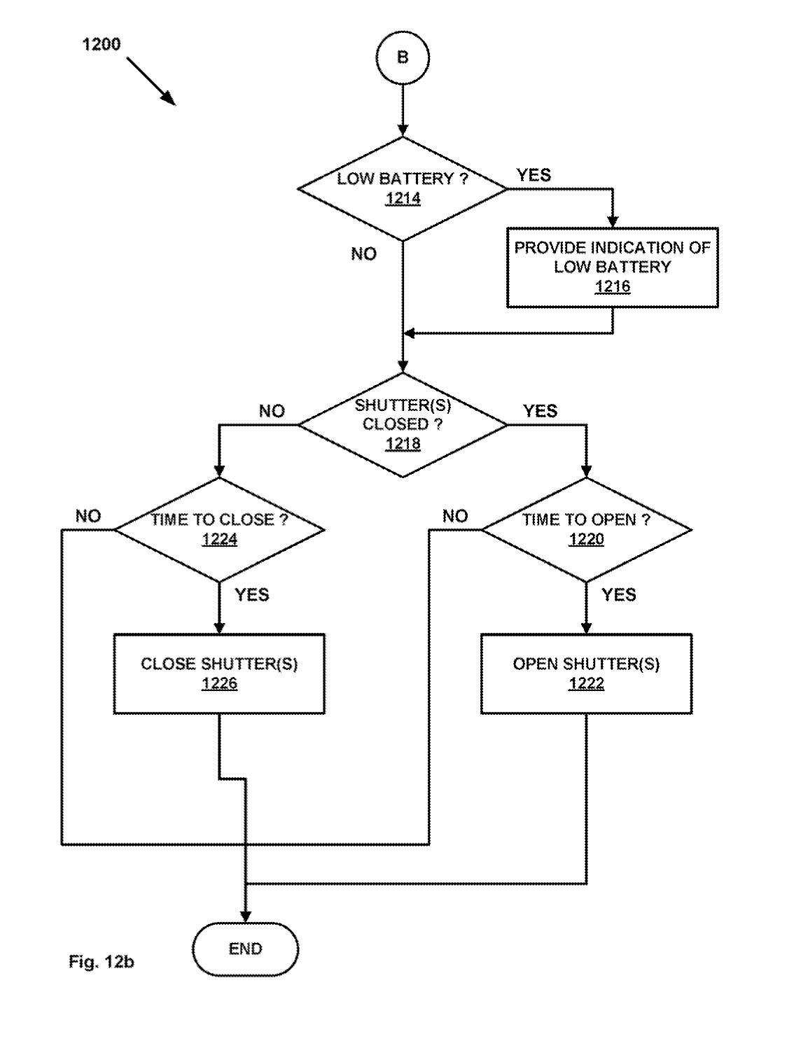

Referring now to FIGS. 12a-12b, in an exemplary embodiment, the 3D glasses 100 implement a method 1200 of servicing one or interrupts that may be provided as part of 1140 in the method 1100.

In 1202, the 3D glasses 100 determine if watchdog timers A or B have expired. If, in 1202, the 3D glasses 100 determine that watchdog timer A has expired, then, in 1204, the 3D glasses determine if the left shutter 120 is active. In an exemplary embodiment the left shutter 120 is active if the 3D glasses 100 are being used to treat Lazy Eye by cycling the left shutter between an occluding state and a non-occluding state.

If the 3D glasses 100 determine in 1204 that the left shutter 120 is active, then, in 1206, the 3D glasses switch the voltage polarity on the left shutter 120. Alternatively, if, 1204, the 3D glasses 100 determine in 1204 that the left shutter 120 is not active, then, in 1208, the 3D glasses determine if the right shutter 122 is active. In an exemplary embodiment the right shutter 122 is active if the 3D glasses 100 are being used to treat Lazy Eye by cycling the right shutter between an occluding state and a non-occluding state.

If the 3D glasses 100 determine in 1208 that the right shutter 122 is active, then, in 1210, the 3D glasses switch the voltage polarity on the right shutter 122.

Alternatively, if the 3D glasses 100 determine in 1208 that the right shutter 122 is not active, then, in 1212, the 3D glasses switch the voltage polarity on both the left and right shutters, 120 and 122.

In an exemplary embodiment, switching the voltage polarities on the left and/or right shutters, 120 and/or 122, extends the operational life of the left and right shutters. In an exemplary embodiment, the left and right shutters, 120 and 122, may be pi cell and/or TN cells.

Alternatively, if, in 1202, the 3D glasses 100 determine that watchdog time B has expired, then, in 1214, the 3D glasses determine if the power level of the battery 110 is low. If the power level of the battery 110 is low, then, in 1216, the 3D glasses 100 will provide a visual indication of the low battery power level to a user of the 3D glasses as described herein.

In 1218, the 3D glasses 100 will determine if the shutters, 120 and/or 122, are closed. In an exemplary embodiment, a shutter is considered closed if the optical transparency of the shutter is at a reduced level as compared with an open state in which the optical transparency of the shutter is at an increased level for purposes of providing contrasting optical transparency for the treatment of Lazy Eye.

If the 3D glasses 100 determine that the shutters, 120 and/or 122, are closed, then, in 1220, the 3D glasses determine if it is now time to open the shutters. In an exemplary embodiment, the time for opening the shutters, 120 and/or 122, will be determined as a function of the time defined for treatment of Lazy Eye in which one or both shutters may cycle between a closed state and an open state. If the 3D glasses 100 determine that it is time to open the shutters, 120 and/or 122, then, in 1222, the 3D glasses will open the shutters, 120 and/or 122.

Alternatively, if the 3D glasses 100 determine that the shutters, 120 and/or 122, are open, then, in 1224, the 3D glasses determine if it is now time to close the shutters. In an exemplary embodiment, the time for closing the shutters, 120 and/or 122, will be determined as a function of the time defined for treatment of Lazy Eye in which one or both shutters may cycle between a closed state and an open state. If the 3D glasses 100 determine that it is time to close the shutters, 120 and/or 122, then, in 1226, the 3D glasses will close the shutters, 120 and/or 122.

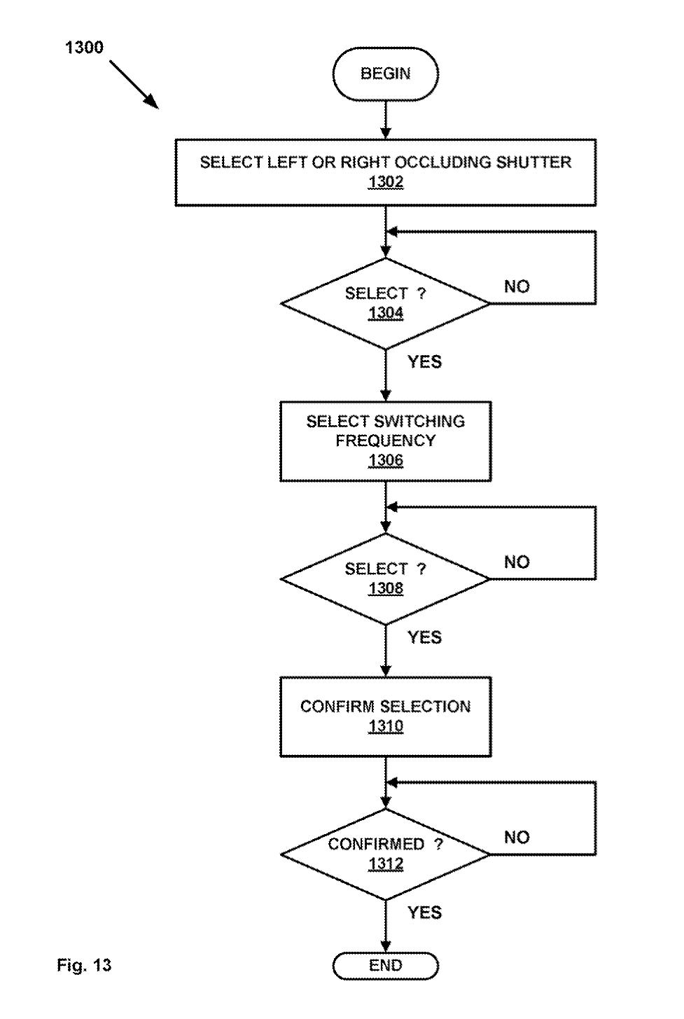

Referring now to FIG. 13, in an exemplary embodiment, the 3D glasses 100 implement a method 1300 of defining the operational characteristics of the 3D glasses when treating Lazy Eye that may be provided as part of 1134 in the method 1100. Furthermore, the method 1300 may be implemented whenever the 3D glasses 100 are connected to one or more host computers 128 and/or wired and/or wireless networks, which may, for example, include the Internet.

In 1302 and 1304, the user may select the left or right shutter, 120 or 122, of the 3D glasses 100 as the occluding shutter for treating Lazy Eye.

Once the user has selected the occluding shutter, then, in 1306 and 1308, the user may select the frequency at which the occluding shutter is cycled between open and closed states.

In 1312, the user may then confirm the selection of the operational parameters to be used in the treatment of Lazy Eye using the 3D glasses. In an exemplary embodiment, the operating parameters selected using the method 1300 are then stored in the memory 106 of the 3D glasses 100. In an exemplary embodiment, the method 1300 may be used by a physician treating a patient who suffers from Lazy Eye and access to the method 1300 may require the entry of a unique patient identifier and a password.

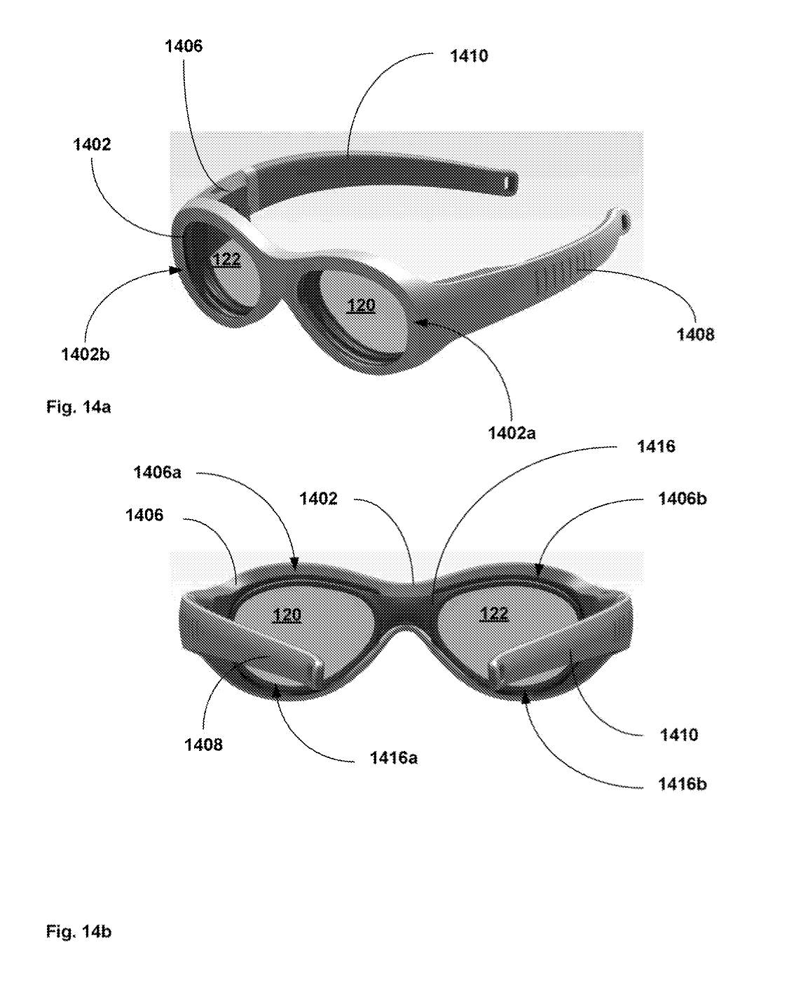

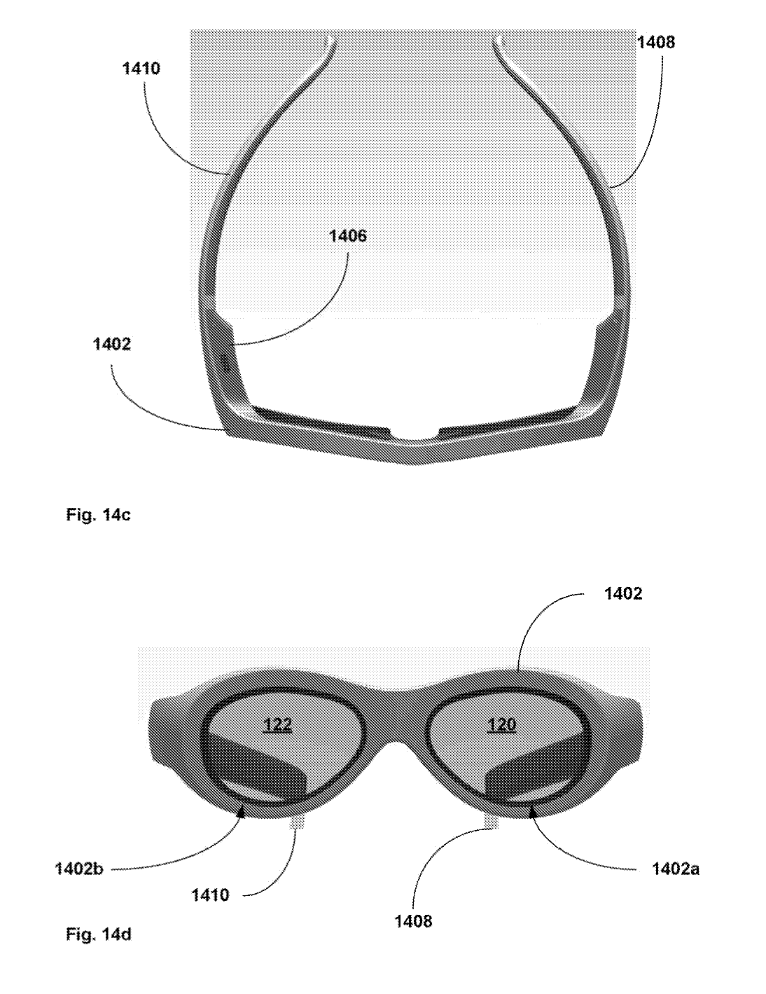

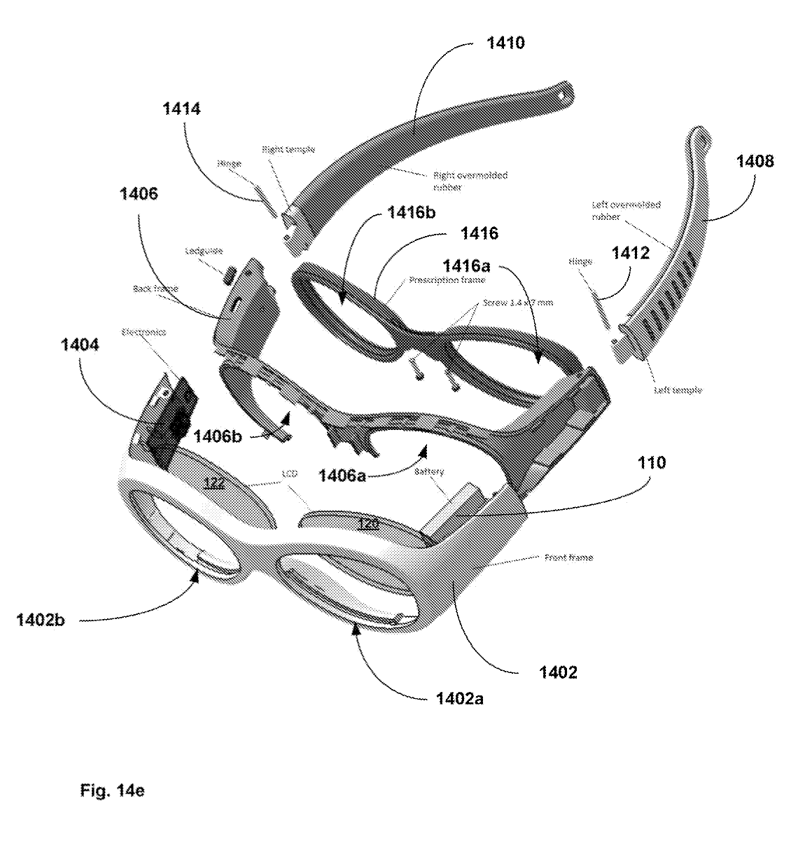

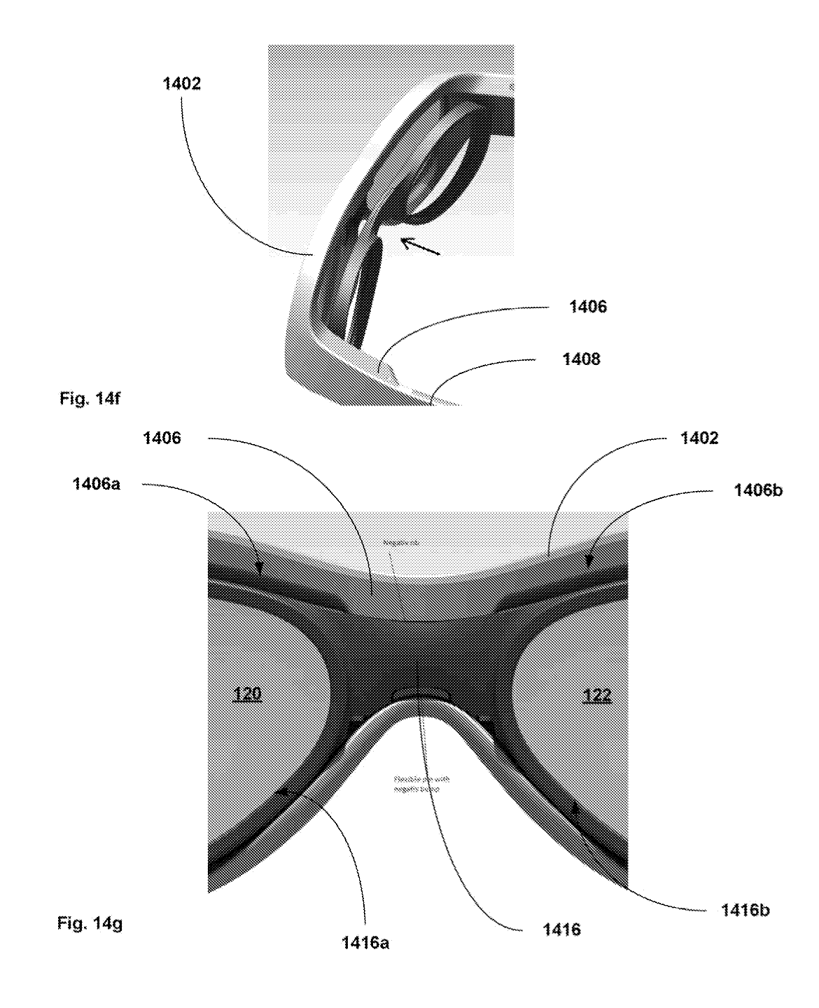

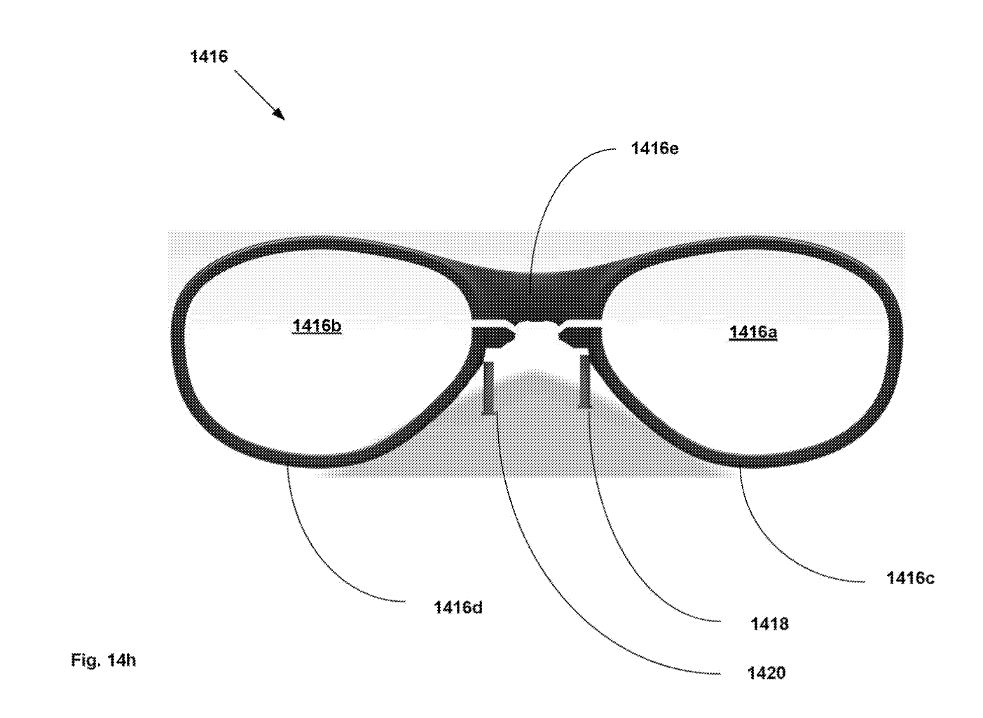

Referring now to FIGS. 14a-14h, an exemplary embodiment of shutter glasses 1400 for treating Lazy Eye will now be described. In an exemplary embodiment, the shutter glasses 1400 are substantially identical in design in operation to the 3D glasses 100 except as described below. Furthermore, the shutter glasses 1400 may implement one or more of the methods described herein.

In an exemplary embodiment, the glasses 1400 include a front frame 1402 that defines openings, 1402a and 1402b, for mounting the left and right shutters, 120 and 122. In an exemplary embodiment, the front frame 1402 further defines one or more internal cavities for mounting and housing the CPU 102, the signal sensor 104, the memory 106, the user interface 108, the battery 110, the left shutter controller 112, the right shutter controller 114, the interface 116, and the display 118. In an exemplary embodiment, the functional elements 1404 of the glasses 1400, which include the CPU 102, the signal sensor 104, the memory 106, the user interface 108, the left shutter controller 112, the right shutter controller 114, the interface 116, and the display 118, are mounted and received within a cavity on one side of the front frame 1402 and the battery 110 is mounted and received within a cavity on another side of the front frame.

In an exemplary embodiment, the glasses 1400 further include a back frame 1406, that mates with and is connected to the front frame 1402, that defines openings, 1406a and 1406b, for mounting the left and right shutters, 120 and 122. In an exemplary embodiment, the back frame 1406 further defines one or more internal cavities for mounting and housing the functional elements 1404 on one side of the rear frame and the battery 110 is mounted and received within a cavity on another side of the front frame.

In an exemplary embodiment, the glasses 1400 further include left and right temples, 1408 and 1410, that are pivotally coupled to the front and rear frames, 1402 and 1406, by corresponding hinges, 1412 and 1414, respectively. In this manner, the temples, 1408 and 1410, may be folded inwardly by the user for easier storage.

In an exemplary embodiment, the glasses 1400 further include a frame 1416 for receiving and mounting prescription lenses that defines openings, 1416a and 1416b, for receiving and mounting the prescription lenses. In an exemplary embodiment, the openings, 1416a and 1416b, of the frame 1416 are defined by elongated arcuate flexible arms, 1416c and 1416d, respectively, that extend from and then circle back toward a central nose piece 1416e. In an exemplary embodiment, the free ends of the arms, 1416c and 1416d, of the frame 1416 may then be secured to the nose piece 1416e by removable fasteners, 1418 and 1420, respectively. In this manner, prescription lenses may be inserted into the openings, 1416a and 1416b, of the frame 1416 and then secured in place using the fasteners, 1418 and 1420, to secure the free ends of the arms, 1416c and 1416d, to the nose piece 1416e.

In an exemplary embodiment, the frame 1416 may then be releasably secured to the back frame 1406 of the glasses 1400, using a conventional releasably connection, thereby positioning the openings, 1416a and 1416b, of the frame parallel and in opposition to the openings, 1402a and 1402b and 1406a and 1406b, of the front and rear frames, 1402 and 1406. In this manner, during operation of the glasses 1400, the user may also use their prescription lenses.

In an exemplary embodiment, one or more aspects of the exemplary apparatus and methods described herein may be applied and implemented in conventional 3D shutter glasses. In this manner, the operation of such conventional 3D shutter glasses may be enhanced thereby. Furthermore, in this manner, such conventional 3D glasses may be operated to provide a treatment for Lazy Eye.

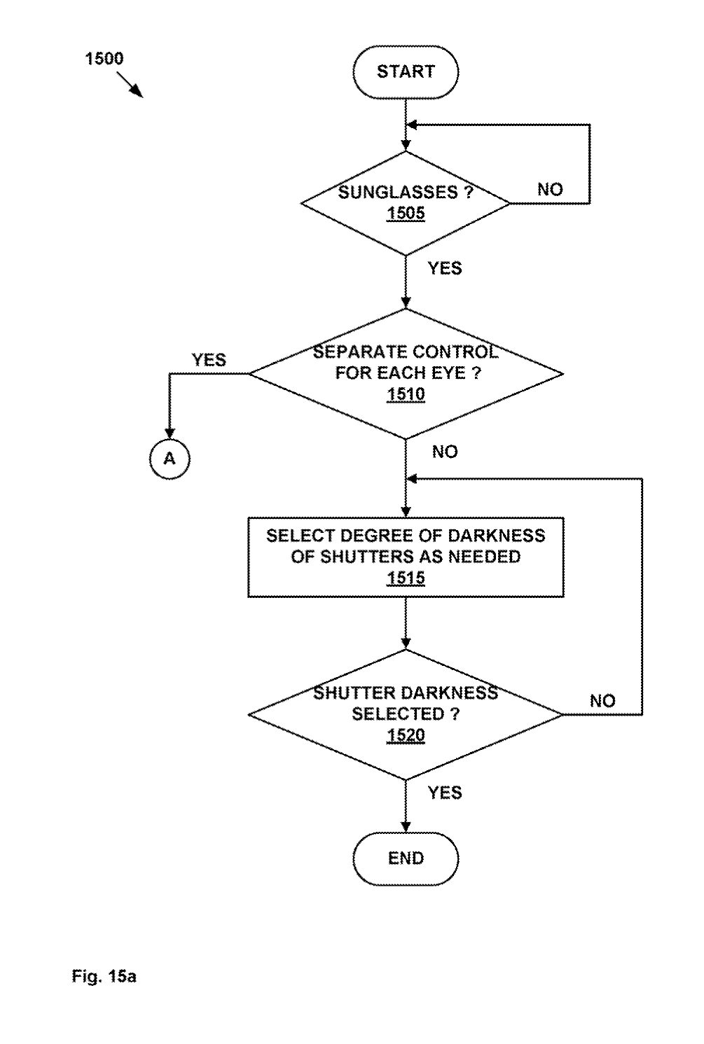

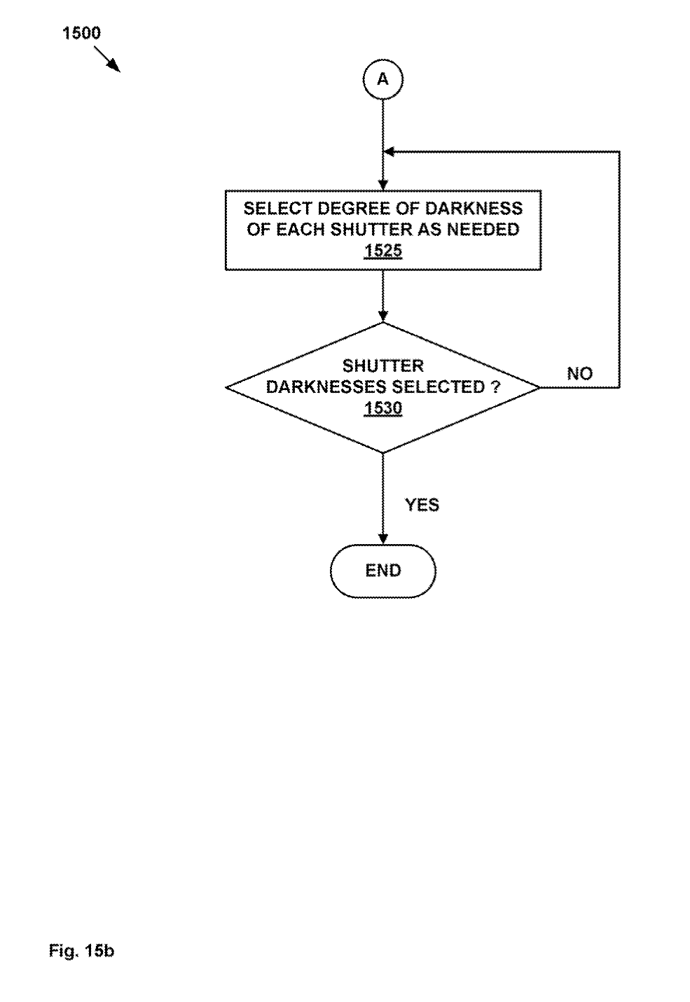

Referring now to FIGS. 15a and 15b, in an exemplary embodiment, the 3D shutter glasses, 100 and/or 1400, implement a method of operation 1500 in which the 3D shutter glasses are operated to provide sunglasses whereby one or both of the shutters are operated to limit the amount of light that passes therethrough by controlling the frequency and/or duration with which one or both of the shutters are optically transmissive.

In an exemplary embodiment, in 1505, the user of the 3D glasses may select operation of the 3D glasses as sunglasses using, for example, a conventional user interface which may include one or more of: push buttons, computer interface, or other user interfaces.

If the user of the 3D glasses selects sunglasses operation, then the user may then select separate control of each of the right and left shutters of the 3D glasses in 1510. In an exemplary embodiment, if the user did not select separate control of each of the left and right shutters, then in 1515 and 1520, the user may select the degree of light passage through each of the shutters.

Alternatively, if the user did select separate control of each of the left and right shutters, then in 1525 and 1530, the user may select the degree of light passage through each of the shutters such that the degree of light passage may be different for each of the shutters.

In an exemplary embodiment, the method 1500 provides controllable darkness sunglasses that may, for example, relieve symptoms of glare, halos, etc. in users who suffer minor optical aberrations. In such users, these minor optical aberrations may be experienced as glares, halos and tails, usually aggravated by bright lights, especially at night, such as by incoming car lights while driving at night. Thus, the present method 1500 may provide relief to such users during daylight and night time operating environments.

Furthermore, in an exemplary embodiment, the method 1500 permits the user to control each shutter separately to allow the eye of the user that may be giving rise to the symptoms associated with one or more optical aberrations to be optically darkened more than the other shutter thereby still maintaining binocular vision in the user.

Furthermore, in an exemplary embodiment, the method 1500 permits the user to control the perceived transparency by controlling the flicker rate of one or both of the shutters. In particular, in an exemplary embodiment, the method 1500 permits the user to control the time durations of fixed transparency periods for one or both of the shutters that may, for example, rapidly change in cycle. In an exemplary embodiment, during the method 1500, one or both of the shutters, may be controlled to rapidly alternate between 70% transparency and 3% transparency. In an exemplary embodiment, during operation of the method 1500, the perceived optical transparency is controlled by changing the ratios between the durations of periods of higher optical transparency and lower optical transparency.

In an exemplary embodiment, during operation of the method 1500, the duty cycle of the periods of higher optical transparency and lower optical transparency may be constant or variable. For example, in an exemplary embodiment, during operation of the method 1500, the time duration of the 70% optical transparency may be longer (e.g. 10 msec) and the time duration of the 3% optical transparency may be shorter (e.g. 3 msec). As a result, in an exemplary embodiment, the overall perceived optical transparency may be in the range of 60%. Furthermore, for example, the overall perceived optical transparency may be reduced by prolonging the time duration of the lower value of optical transparency, i.e., 3% optical transparency (i.e. to 10 msec), and/or by shortening the time duration of the higher value of optical transparency, i.e., 70% optical transparency (i.e. to 3 msec). In some exemplary embodiments, during operation of the method 1500, the sum of both periods, i.e., the low optical transparency and high optical transparency time durations, is always fixed, and may, for example, be in the range of 10 msec to 50 msec.

In an exemplary embodiment, during operation of the method 1500, the overall optical transparency perceived by the user may be varying one or more of: a) the duty cycle; b) the duty cycle driving waveform shapes, c) the optical transparency of the liquid crystal materials during the lower and higher optical transparency portions of the duty cycle; and/or d) the overall frame rate of the duty cycle (e.g., 60 Hz).

In an exemplary embodiment, the method 1500 may be used to provide sunglasses using a conventional pair of 3D shutter glasses programmed with the operations of the method 1500.

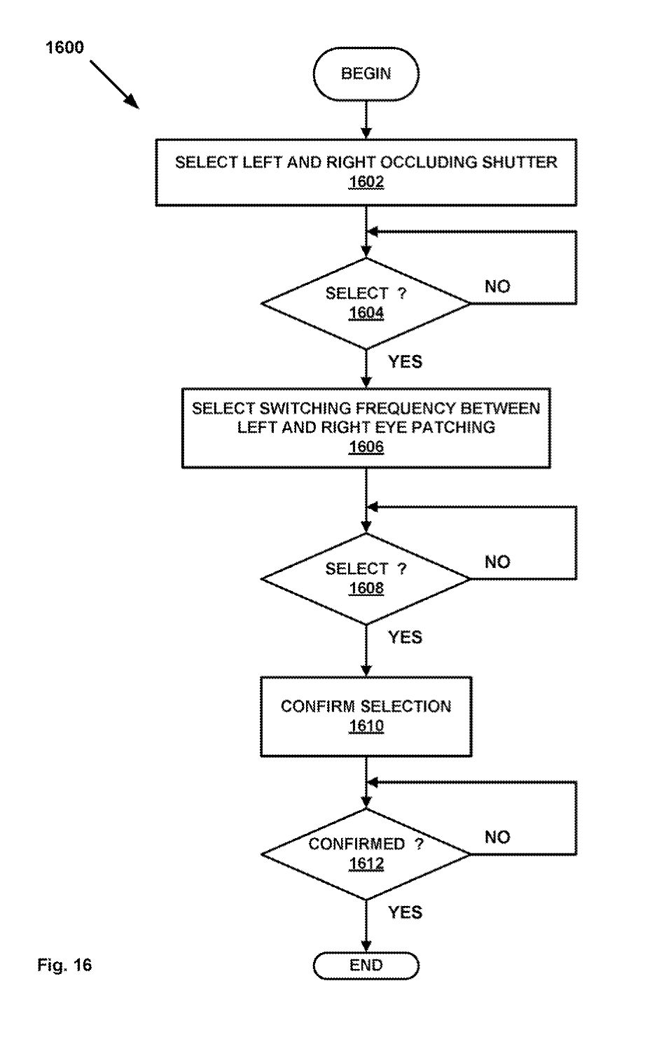

Referring now to FIG. 16, in an exemplary embodiment, the 3D shutter glasses, 100 and/or 1400, implement a method of operation 1600 in which the 3D shutter glasses are operated to provide alternate eye patching (i.e., optically covering one eye of a user at a time, alternating between the eyes of the user)—to treat Intermittent Exotropia.

In an exemplary embodiment, the method 1600 may, for example, be implanted as part of 1134 in the method 1100 and/or as part of the method 1300. Furthermore, in an exemplary embodiment, the method 1600 may be implemented whenever the 3D shutter glasses are connected to one or more host computers 128 and/or wired and/or wireless networks, which may, for example, include the Internet.

In 1602 and 1604, the user may select using the left and right shutter, 120 or 122, of the 3D shutter glasses 100 as the occluding shutters for treating Intermittent Exotropia.

Once the user has selected using both of the shutters, on an alternating basis, as the occluding shutter, then, in 1606 and 1608, the user may select the frequency at which the occluding shutters are used, in the alternative, as the occluding shutters for treating Intermittent Exotropia.

In 1610 and 1612, the user may then confirm the selection of the operational parameters to be used in the treatment of Intermittent Exotropia using the 3D shutter glasses. In an exemplary embodiment, the operating parameters selected using the method 1600 are then stored in the memory 106 of the 3D shutter glasses 100. In an exemplary embodiment, the method 1600 may be used by a physician treating a patient who suffers from Intermittent Exotropia and access to the method 1600 may require the entry of a unique patient identifier and a password.

In an exemplary embodiment, the method 1600 may be provided by programming a conventional pair of 3D shutter glasses.

In an exemplary embodiment, one or more of the methods of the present disclosure may be implemented by a physician and/or an optician. Thus, one of the advantages of using 3D shutter glasses to provide electronic patching of the eyes of a user is the ability to allow the physician and/or the optician to program the operation of the shutter glasses at will. Just as currently done with conventional physical patching of eyes, some physicians prefer to initiate the therapy with only mild occlusions, while some other physicians prefer to start with more aggressive occlusion as some patients will need light occlusions only while some other patients will need more hours of occlusion. In addition, even the same physician treating Amblyopia or Intermittent Exotropia in the same patients will often want to change the occlusion patterns with time.

In an exemplary embodiment, one or more of the methods of the present disclosure for controlling the operation of the 3D shutter glasses, 100 and/or 1400, may be implemented by providing a conventional push button as the user interface.

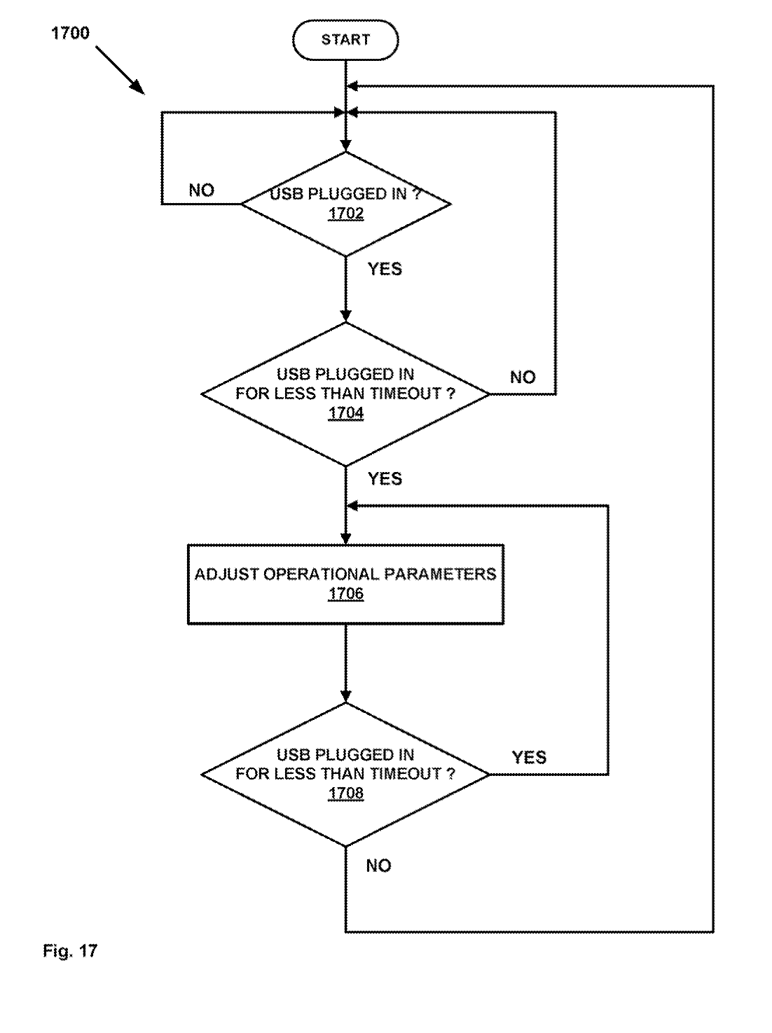

In particular, referring to FIG. 17, in an exemplary embodiment, a method 1700 of setting the operating parameters of the shutter glasses, 100 and/or 1400 and/or conventional shutter glasses, may be used in which, in 1702, it is determined if the shutter glasses are connected to a power source such as, for example, a USB connection, or something equivalent thereto.

If the shutter glasses are connected to a power source, then it is determined if the shutter glasses have been connected to the power source for less than a predetermined timeout period in 1704. If the shutter glasses have been connected to the external power source for less than the predetermined timeout period, then the user may adjust one or more of the operational parameters of the shutter glasses in 1706 and 1708 by manipulating one or more user interfaces such as, for example, a push button, or equivalent device, provided on the shutter glasses. So long as the shutter glasses have been connected to the power source for less than the predetermined timeout period, then the user may continue to adjust one or more of the operating parameters of the shutter glasses. In this manner, the method 1700 minimizes the possibility of accidental and/or unwanted changes to the operational parameters of the shutter glasses. In several alternative embodiments, the detection of an RFID device or magnet proximate the shutter glasses may be used instead of, or in addition to, the monitoring the timeout periods in 1704 and 1708.

In several exemplary embodiments, during the operation of the method 1700, in 1708, the operational parameters that may be adjusted may, for example, include operational parameters for using the shutter glasses to view 3D images, using the shutters glasses to treat Amblyopia, or using the shutter glasses to treat other medical conditions.

In an exemplary embodiment, using the method 1700, in 1708, the user may adjust the operational parameters of the shutter glasses for using the shutter glasses, 100 and/or 1400 and/or conventional shutter glasses, to treat Amblyopia such that each depression of a push button on the shutter glasses for a predetermined time period will then sequence the shutter glasses between different and distinct operational modes.

For example, in an exemplary embodiment, using the method 1700, in 1708, the eye of the user that will be electronically patched by operation of the shutter glasses is preset and can only be changed by reprogramming the firmware within the shutter glasses and cannot be changed by the user of the shutter glasses. Operation of the shutter glasses in this exemplary embodiment initiates at a default operational mode in which the selected eye of the user is treated using the following duty cycle: 20 seconds of occlusion, followed by 80 seconds open state. The user, or preferably the physician or optician, may then select from a limited number of alternative operational modes by continually depressing the push button for a predetermined minimum time period to thereby select the alternative operational modes. In an exemplary embodiment, the additional alternative operational modes include the following: 1) 20 seconds of occlusion, followed by 40 seconds open state; and 2) 30 seconds of occlusion, followed by 30 seconds open state.

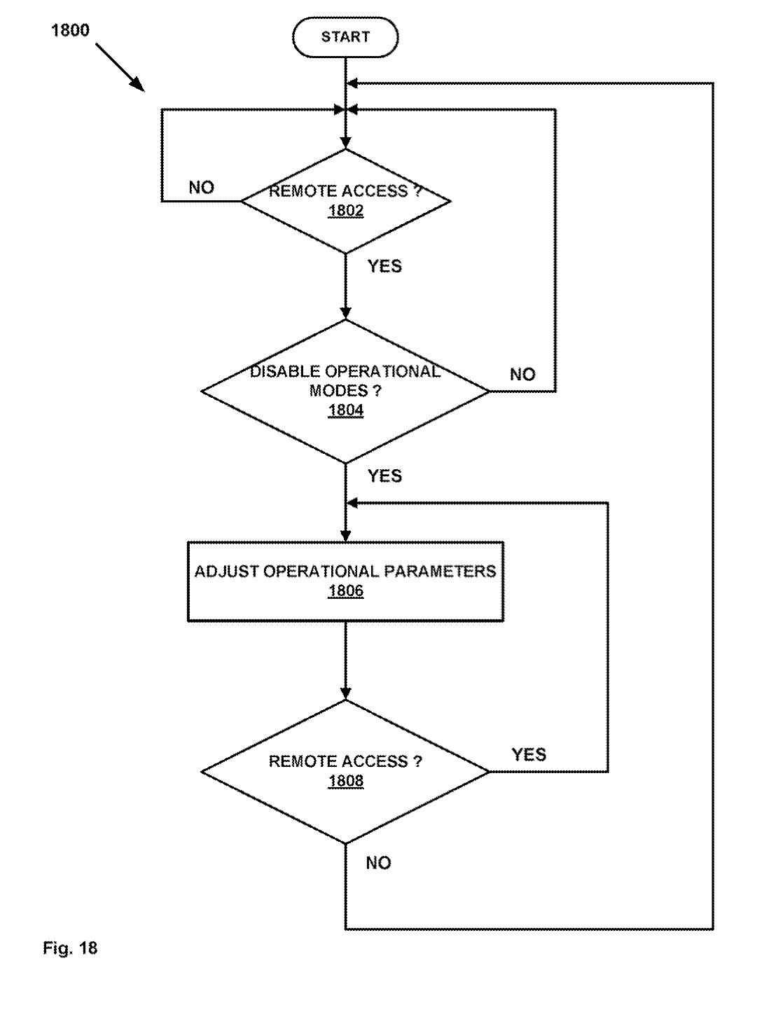

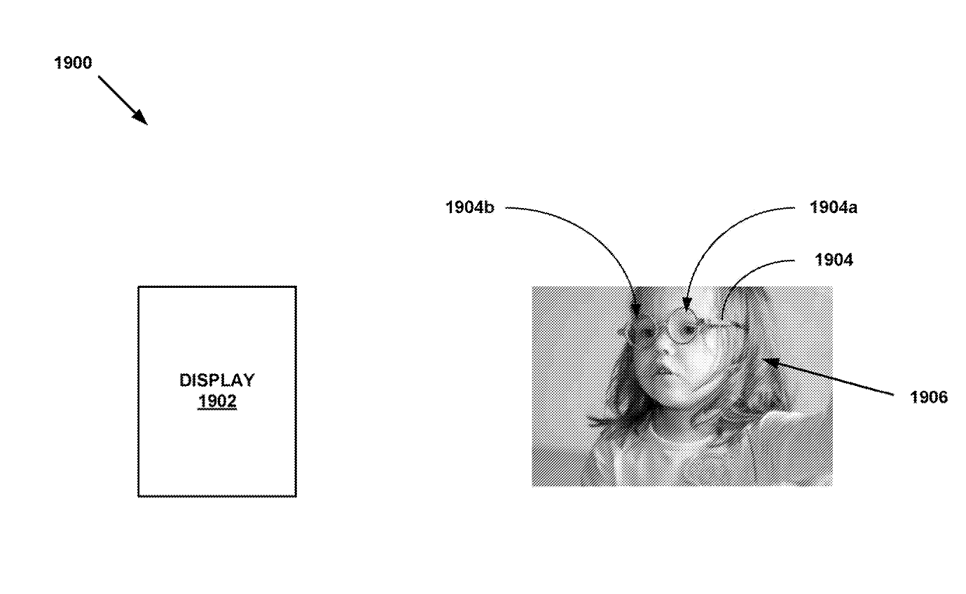

In an exemplary embodiment, referring now to FIG. 18, the shutter glasses, 100 and/or 1400 and/or conventional shutter glasses, may implement a method 1800 of providing parental control in which, in 1802, the parent of the user may remotely access the shutter glasses using a wireless interface such as, for example, RF, Bluetooth, IR or other equivalent wireless interface. In 1804 and 1806, the parent may then, using a password protected interface, then adjust or disable one or more of the operational parameters of the shutter glasses.

In this manner, in an exemplary embodiment, the method 1800 permits the parent to disable the operation of the shutter glasses to treat Amblyopia, i.e., stopping any and all occlusions of the eyes of the child. As a result, the child user of the shutter glasses may be then focus on other activities that are visual field dependent such as, for example, sports, bicycling, or other similar activities.

In an exemplary embodiment, one or more portions of the methods of the present disclosure may be implemented by permitting the user of the shutter glasses to adjust one or more of the operating parameters by a single touch of pressure sensitive portions of the shutter glasses.

In an exemplary embodiment, one or more of the methods of the present disclosure include the shutter glasses: 1) sensing and recording the amount time that the user watches a display device such as, for example, a television; 2) sensing the amount of ambient lighting and turning the glasses on or off as a function of the amount of ambient lighting; 3) sensing the placement of the glasses on the head of a user; and 4) recording the amount and degree of therapy provided to the user of the glasses.

In an exemplary embodiment, referring to FIG. 19, a system 1900 for treating Amblyopia includes a conventional display 1902 that generates polarized output light during operation. In an exemplary embodiment, the display 1902 may be a television display, a computer display, a smart phone display, or other display device having output light that is polarized in some manner.

A pair of glasses 1904 with left and right lenses, 1904a and 1904b, are then worn by a user 1906 while the user watches the display 1902. In an exemplary embodiment, one of the eyes of user 1906 require treatment for Amblyopia. As a result, the corresponding lens of the glasses 1904 is polarized such that the polarized light output from the display 1902 is prevented, or at least attenuated, from passing therethrough to the stronger eye of the user 1906 that is being passively patched by operation of the polarizer in the lens of the glasses. The other lens of the glasses 1904, positioned over the weaker eye of the user 1906, is optically clear to permit substantially all of the light output of the display 1902 to reach the weaker eye.

Thus, the system 1900 permits the user 1906 to receive passive treatment for Amblyopia while watching a display device such as, for example, a television or video player or gaming console. As a result, the user 1906 will probably enjoy the treatment for Amblyopia, even if conducted for a long time duration.

In an exemplary embodiment, during the operation of one or more of the methods of the present application to treat Lazy Eye, the methods may further incorporate one or more of the apparatus and methods disclosed in U.S. Pat. No. 5,452,026, the disclosure of which is incorporated herein by reference.

It is understood that variations may be made in the above without departing from the scope of the invention. While specific embodiments have been shown and described, modifications can be made by one skilled in the art without departing from the spirit or teaching of this invention. The embodiments as described are exemplary only and are not limiting. Many variations and modifications are possible and are within the scope of the invention. Furthermore, one or more elements of the exemplary embodiments may be omitted, combined with, or substituted for, in whole or in part, one or more elements of one or more of the other exemplary embodiments. Accordingly, the scope of protection is not limited to the embodiments described, but is only limited by the claims that follow, the scope of which shall include all equivalents of the subject matter of the claims.

What is claimed is:

1. A viewing system, comprising:
an eyewear device used for treatment of a user, the eyewear device comprising a first lens having a first liquid crystal shutter and a second lens having a second liquid crystal shutter;
a first controller operably coupled to the first liquid crystal shutter for controlling a degree of optical transmissivity of the first liquid crystal shutter at a first predetermined rate;
a second controller operably coupled to the second liquid crystal shutter for controlling the degree of optical transmissivity of the second liquid crystal shutter at a second predetermined rate;
a memory operably coupled to the first controller and the second controller for storing an operating parameter for the eyewear device; and
an operating interface operably coupled to the first controller, the second controller, the memory, and a conventional interface device for permitting the operating parameter of the eyewear device to be defined and stored in the memory.

2. The system of claim 1, further comprising a frame for holding prescription lenses that are adapted to be removably coupled to the eyewear device.

3. The system of claim 1, further comprising an RF sensor operably coupled to one or more of the first controller or the second controller for sensing an RF signal transmitted to the eyewear device.

4. The system of claim 1, further comprising an IR sensor operably coupled to one or more of the first controller or the second controller for sensing an IR signal transmitted to the eyewear device.

5. The system of claim 1, further comprising a user interface operably coupled to one or more of the first controller or the second controller.

6. The system of claim 5, wherein the user interface comprises one or more a push button, a touch screen, a display, or an indicator light for one or more indicating or adjusting an operating mode of the eyewear device.

7. (canceled)

8. (canceled)

8a. (canceled)

8b. (canceled)

8c. (canceled)

9. A method for treating eyes of a user, the method comprising:
storing within a memory of an eyewear device a first predetermined frequency at which a first liquid crystal shutter of the eyewear device will be opened and closed while a second liquid crystal shutter remains open, and a second predetermined frequency at which the second liquid crystal shutter will be opened and closed while the first liquid crystal shutter remains open;
opening and closing the first liquid crystal shutter at the first predetermined frequency while keeping the second liquid crystal shutter opened;
opening and closing the second liquid crystal shutter at the second predetermined frequency while keeping the first crystal shutter opened; and
adjusting an operating parameter of the eyewear device by interfacing the eyewear device with one or more of a first controller for controlling the first liquid crystal shutter, a second controller for controlling the second liquid crystal shutter, a memory, an operating interface, or a conventional interface device, wherein the operating parameter is changed in response to user input.

10. The method of claim 9, further comprising:
selecting an amount of light allowed to pass through one or more of the first liquid crystal shutter and the second liquid crystal shutter; and
adjusting one or more of the first liquid crystal shutter or the second liquid crystal shutter in response to the selected amount of light allowed.

11. (canceled)

12. (canceled)

13. (canceled)

14. (canceled)

15. (canceled)

16. The method of claim 9, further comprising coupling the operating interface to an external power source.

17. The method of claim 16, further comprising determining that the eyewear device has been connected to the external power source for less than a predetermined timeout period.

18. (canceled)

19. (canceled)

20. (canceled)

21. The system of claim 1, wherein the conventional interface device comprises one or more of a host computer, a smart phone, a personal digital assistant, an external battery, a USB connected device, a WiFi connected device, a Bluetooth connected device, a wired interface connected device, or a wireless interface connected device.

22. The system of claim 1, wherein the operating parameter includes one or more of parameters for:
repetitively occluding a stronger eye of the user with the eyewear device at one or more of the first or second predetermined frequencies while providing a transparent view for the weaker eye of the user with the eyewear device at one or more of the first or second predetermined frequencies to treat Amblyopia, Intermittent Exotropia, or other occular disorder of the user;
operating the eyewear device to provide protection from ambient light; or
treating Amblyopia, Intermittent Exotropia, or other ocular disorder of the user with alternatingly covering of one eye of the user at a time.

23. The system of claim 1, wherein the operating interface permits changes to the operating parameter by remote access with a password.

24. The system of claim 5, further comprising one or more shades removably coupled to the eyewear device over one or more of the first or second liquid crystal shutters to permit the user to select a degree of light passage through one or more the first liquid crystal shutter or the second liquid crystal shutter.

25. The method of claim 9, wherein the operating parameter is provided by the user.

26. The method of claim 25, further comprising repetitively occluding a stronger eye of the user with the eyewear device at one or more of the first or second predetermined frequencies while providing a transparent view for the weaker eye of the user with the eyewear device at one or more of the first or second predetermined frequencies to treat Amblyopia, Intermittent Exotropia, or other occular disorder of the user.

27. The method of claim 9, further comprising alternatingly covering one eye of the user at a time to treat Amblyopia, Intermittent Exotropia, or other occular disorder of the user.

28. The method of claim 9, further comprising permitting changes to the operating parameter by providing remote access to the operating interface with a password.

29. The method of claim 9, wherein the conventional interface device comprises one or more of a host computer, a smart phone, a personal digital assistant, an external battery, a USB connected device, a WiFi connected device, a Bluetooth connected device, a wired interface connected device, or a wireless interface connected device.

30. The method of claim 9, further comprising permitting the user to limitedly adjust the operating parameter by interfacing with one or more of the first controller, the second controller, the memory, the operating interface, the conventional interface device, or a user interface of the eyewear device to minimize the possibility of accidental and unwanted changes to the operating parameter.
